(19) United States
(12) Patent Application Publication
(10) Pub. No.: US 2007/0269452 A1
(43) Pub. Date: Nov. 22, 2007

(54) Methods and compositions for the diagnosis and treatment of cancer

(76) Inventors: Joel Yisraeli, Efrat (IL); Eli Pikarsky, Jerusalem (IL); Gilad Vainer, Carmiel (IL); Gail Amir, Jerusalem (IL)

Correspondence Address: WINSTON & STRAWN LLP;PATENT DEPARTMENT, 1700 K STREET, N.W., WASHINGTON, DC, 20006, US

(21) Appl. No.: 11/729,515

(22) Filed: Mar. 28, 2007

Related U.S. Application Data
(63) Continuation of application No. PCT/IL05/01060, filed on Oct. 2, 2005.
(60) Provisional application No. 60/615,202, filed on Oct. 4, 2004.
(60) Provisional application No. 60/614,969, filed on Oct. 4, 2004.

(51) Int. Cl. A61K 38/00; A61K 39/00; A61K 48/00; A61P 43/00; G01N 33/53

(52) U.S. Cl. 424/185.1; 424/93.1; 435/7.1; 514/2; 514/44

(57) ABSTRACT

The present invention relates to compositions and methods useful for the diagnosis of lymphoma and particular types of metastatic tumors and for treating same. Specifically, this invention relates to methods for the differential diagnosis of B cell derived lymphoma subtypes and to the diagnosis of the metastatic potential of some types of tumors by detecting at least one member of the VICKZ family in suspect tissue. The present invention further relates to kits for the detection of VICKZ expression and to therapeutic compositions and methods for treating B cell derived lymphoma subtypes and certain types of metastatic disease.

### CROSS-REFERENCE TO RELATED APPLICATIONS

This application is a continuation of International application PCT/IL2005/001060 filed Oct. 2, 2005, and which claims the benefit of provisional applications 60/615,202 and 60/614,969, each filed Oct. 4, 2004. The entire content of each application is expressly incorporated herein by reference thereto.

### FIELD OF THE INVENTION

The present invention relates to compositions and methods useful for the diagnosis of lymphoma and particular types of metastatic tumors and for treating same. Specifically, this invention relates to methods for the differential diagnosis of B cell derived lymphoma subtypes and for diagnosis of the metastatic potential of tumors by detecting at least one member of the VICKZ family in suspect tissue.

### BACKGROUND OF THE INVENTION

VICKZ Proteins

VICKZ proteins are a highly conserved family of RNA binding proteins (RBP). “VICKZ” is an acronym of the first letters of the founding members of this family (VgI RBP (VgI RNA binding protein), IMP (IGF-II mRNA-binding protein), CRD-BP (c-myc coding region determinant binding protein), KOC (KH-domain containing protein over expressed in cancer), ZBP-I (zipcode binding protein)). Each member of the family has two N-terminal RNA recognition motifs, an RGG RNA binding domain and four C-terminal hnRNP K-homology (KH) domains (Yaniv and Yisraeli, 2002).

In humans, there are three VICKZ protein isoforms encoded on separate chromosomes. The VICKZ proteins have been classified into three distinct subfamilies based on homology to each of three different human homologs. The human proteins VICKZ1, VICKZ2 and VICKZ3, also known as IMP1, IMP2 and IMP3 respectively, are expressed or amplified in certain tumors and have been shown to be essentially absent in normal adult tissue (reviewed in Yaniv and Yisraeli, 2002). These proteins have been classified as “oncofetal” proteins due to their high expression in embryonic tissue and overexpression in certain tumors (Doyle et al, 1998).

VICKZ proteins have been implicated in different aspects of RNA regulation: intracellular localization (Havin et al., 1998), translational repression, and stability (Doyle et al., 1998). A number of studies have identified one or more of the VICKZ proteins as overexpressed in different kinds of cancers (Doyle et al., 1998; Zhang et al, 1999). For example, KOC expression was shown to be an indicator of malignant disease (Mueller et al., 2003) and a molecular marker able to distinguish between benign and malignant pancreatic lesions (Yantiss et al, 2005).

US Patent Application Publication Nos. 20050142620, 20040235072 and related applications teach compositions and methods for the therapy and diagnosis of lung cancer. Among the genes taught in those applications is lung tumor antigen L523S, identified as KOC or VICKZ1. Those applications neither teach nor suggest the use of a VICKZ molecule for either the differential diagnosis of lymphoma or for identifying metastatic tumors or metastases.

Lymphoma

Solid tissue neoplasias of lymphoid cells of the immune system are termed lymphomas. Lymphomas can originate from lymphoid cells at almost any stage of B-cell development, thus giving rise to many different types of lymphoproliferative diseases. Common subtypes of lymphoma include Hodgkin's lymphoma (HL), and non-Hodgkin's lymphomas (NHL) including follicular lymphoma (FL) and diffuse large B-cell lymphoma (DLBCL).

The differential diagnosis and grading of lymphomas is of primary importance to precisely tailor the appropriate treatment.

Lymphomas have been found to maintain the characteristics of the location and developmental stage from which they originate. For example, a Germinal Center (GC) B-cell in the lymph node that undergoes a neoplastic chromosomal translocation will generally continue to express the molecular markers characteristic of non-neoplastic GC B-cells. Thus, it has been possible to classify lymphomas based on the set of markers they express.

Differential diagnosis (DD) is an integral factor in determining treatment and, in many cases, prognosis. The most common types of non-Hodgkin's lymphoma, follicular lymphoma (FL) and diffuse large B-cell lymphoma (DLBCL), account for more than 70,000 new cases per year. FL and DLBCL have been considered to be of GC origin, although recent microarray analyses indicate that a subgroup of DLBCL has the gene-expression signature of an activated B-cell (ABC), thought to be a post-GC stage based on the presence of somatic mutations in the hypervariable region of the immunoglobulin genes (Alizadeh et al., 2000). In contrast to the DLBCL subgroup with the GC-like gene expression signature, DLBCL patients expressing the ABC-like have a much lower survival expectancy following treatment (Alizadeh et al., 2000). The Reed-Stemberg and Hodgkin cells in HL have also recently been shown to be of GC origin (reviewed in Kuppers, 2002).

The identification of cell surface markers on B-cell lymphomas has been useful in the development of reagents for both the diagnosis and treatment of lymphomas. Antibodies directed to specific B-cell surface antigens, including to CD20 (rituximab Rituxan®), CD22 (epratuzumab) and CD74 (hLL1), have been shown to be effective in diagnosing and treating certain lymphomas.

U.S. Pat. No. 6,399,061 teaches a method for depleting peripheral B cells in a lymphoma patient comprising administering an amount of the anti-CD20 antibody sufficient to induce B cell depletion. The CD20 cell surface marker is expressed in a broad pattern in both normal and malignant B-cells. U.S. Pat. No. 5,407,805 discloses a monoclonal antibody produced by the TG-1G9 hybridoma cell line, useful for the diagnosis and therapy of various leukemias and lymphomas. PCT patent publication WO 96/04925 teaches a chimeric and humanized LL2 monoclonal antibody for use in diagnosing and treating B-cell lymphomas and leukemias. There are no teachings of VICKZ as a marker in B-cell derived lymphomas. Furthermore, the art neither teaches nor suggests the use of VICKZ for the differential diagnosis of lymphoma subtypes or the use of VICKZ modulators for treating B-cell derived lymphoma.

Metastatic Disease

Metastatic disease is the spread of cancer from a primary focus to one or more secondary points in the body. In order to metastasize, a tumor cell must mobilize itself into the circulatory system in a process referred to as intravasation. During a process known as extravasation, the tumor cell leaves the blood circulation, and penetrates the host tissue, again crossing through a basement membrane. The tumor cells that survive this process and are able to grow in an ectopic environment form clinically significant metastases that pose a life-threatening situation to the host. The ability to form tumor metastases is characteristic of highly malignant cancers with poor clinical outcome.

A correlation between the metastatic potential of tumors and the degree of VICKZ expression was hypothesized based on the observations that VICKZ1 binds β-actin RNA and appears to shuttle it to the leading edge of migrating cells and that VICKZ is overexpressed in certain cancers (Yaniv and Yisraeli, 2002). However, this document provides no guidance for distinguishing malignant cancers and, in particular, those having high metastatic potential. Furthermore, there have been inconsistent findings concerning a correlation between VICKZ expression and metastases or metastatic potential of a tumor.

In one study, ZBP-I expression, as measured by QRT-PCR, was followed in tumors originating from rat mammary adenocarcinoma MTLn3 and MTC cell lines, having high metastatic and low metastatic potential, respectively. Both the MTLn3 cultured cells and the tumors derived from that highly metastatic cell line expressed much lower levels of ZBP-I than the MTC cultured cells or MTC derived tumors. In an earlier study, the MTLn3 cells show no peripheral β-actin RNA localization while the non-metastatic MTC cell line localized β-actin RNA to the leading edge of the cell (Shestakova et al, 1999). It has recently been postulated that ZBPI is a candidate for a “metastatic repressor” and together with mRNA targeting and analysis of tumour cell polarity around blood vessels may be used in prognosis” (Condeelis and Singer, 2005).

Colorectal Cancers

Colorectal cancer (CRC) also known as colon cancer, colon carcinoma, colorectal carcinoma and adenocarcinoma of the colon, accounts for over 55,000 deaths a year in the U.S. alone. The developmental stages of the large majority of these cancers have been described in detail, beginning with non-neoplastic polyps that appear to develop into neoplastic epithelial lesions. These growths can invade adjacent structures and metastasize through the lymphatics and blood vessels to distal sites, most frequently the liver, lymph nodes and lungs. Underlying mechanisms for metastasis are not well understood, although molecules involved in actin remodeling have been implicated. Metastases are correlated with a poor prognosis and the ability to predict the metastatic potential would be beneficial in determining treatment options for the patient.

No correlation between VICKZ expression and colorectal cancer metastases or metastatic potential is known. Specifically, Ross et al (Ross et al, 2001) shows that CRD-BP (VICKZ1) is expressed in moderately differentiated adenocarcinomas of the colon yet some CRD-BP positive tumors had not metastasized while two CRD-BP negative tumors had metastasized.

U.S. Pat. No. 6,255,055 discloses a method of diagnosing cancer in a human patient by detecting CRD-BP (VICKZ1) levels in human tissue and a method of inhibiting cancer by lowering the CRD-BP level in cancerous tissues. That disclosure teaches that CRD-BP is detected in breast cancer, colon cancer and pancreatic cancer and is not detected in human leukemia. There is no disclosure of CRD-BP expression in metastatic disease. U.S. Pat. No. 6,794,151 teaches a method of detecting breast cancer in a serum sample from a patient using CRD-BP.

The art neither teaches nor suggests the detection of VICKZ expression useful in diagnosing metastatic disease or methods of treating metastatic disease using VICKZ modulators.

### SUMMARY OF THE INVENTION

The present invention provides, for the first time, compositions and methods for the differential diagnosis and treatment of lymphoma subtypes. Until now it was believed that members of the VICKZ family of proteins were “oncofetal” proteins, i.e. expressed in embryonic and neoplastic tissue but weakly expressed or absent in normal tissue. The present invention is based on the unexpected discovery that VICKZ protein is specifically expressed in the cytoplasm of centroblasts and centrocytes, two types of B cells present in Germinal Centers (GC) of normal lymph nodes. The present invention discloses for the first time compositions and methods useful for the detection of VICKZ proteins and RNA in Germinal Centers advantageous in the diagnosis of lymphoma and in particular in the differential diagnosis of B-cell lymphoma subtypes having a GC origin. The present invention further relates to the prevention, attenuation or treatment of GC B-cell derived lymphomas, by reducing the amount or activity of VICKZ expression products or by eliciting an immune response against these products.

Furthermore, VICKZ protein was surprisingly shown to be a marker of metastatic disease, in contradistinction to the known art, and can be used to determine the metastatic potential of a tumor and to treat same. In particular, VICKZ expression in tumor cells or tissue is an indicator of the metastatic potential of that tumor.

The present invention is based on the unexpected discovery that VICKZ proteins are expressed in both in specific B cells and in tumor cells having metastatic potential.

Specifically VICKZ is expressed in the germinal center (GC) of lymph nodes and can be used in the differential diagnosis and treatment of lymphomas. In one aspect the present invention provides methods and kits for detecting the presence and or amount of VICKZ expression products, in particular cells and tissues that express VICKZ polynucleotides and polypeptides. Some embodiments of this invention provide methods for monitoring VICKZ expression in a bodily biological specimen obtained from a subject having or suspected of having cancer selected from the group consisting of germinal center B cell derived lymphoma and metastatic disease. The monitoring may be for the purpose of disease detection, establishing the prognostic course of the disease, for determining the success of various therapeutic regimes, or for establishing admission criteria of a specific patient to a specific therapeutic regime.

In one embodiment the present invention provides a method for the diagnosis of lymphoma in a subject, the method comprising detecting VICKZ expression in a suitable biological specimen from the subject. Preferably the method is for the differential diagnosis of lymphoma subtypes having a GC origin.

The presence of a VICKZ expression product (protein, mRNA) in a biological sample is indicative that the individual is suffering from lymphoma, preferably germinal center B cell derived lymphoma. Examples of GC B cell derived lymphomas include, inter alia, follicular lymphoma, diffuse large B cell lymphoma, Hodgkin's lymphoma and Burkitt lymphoma.

In one embodiment the present invention provides a method for the diagnosis of lymphoma subtypes in a subject, the method comprising the steps of
a) contacting a biological specimen from the subject with a detecting molecule having specific affinity to a VICKZ expression product; and
b) detecting whether the molecule binds to the specimen;

wherein detection of binding between said specimen and said molecule indicates a positive diagnosis of lymphoma. In certain embodiments the above method detects GC B-cell derived lymphoma.

The expression product can be a protein or fragment thereof, in which case the detecting molecule includes antibodies having specific affinity for at least one member of the VICKZ family of proteins. Alternatively, the expression product is a VICKZ specific gene transcript in which case the detecting molecule includes a nucleic acid probe having a sequence complementary to at least a part of the RNA or a cDNA transcribed therefrom.

In another embodiment the present invention provides a method for the diagnosis of metastatic disease in a subject. In some embodiments the method is an in vitro method performed on a biological specimen obtained from the subject. In some embodiments metastatic disease is selected from the group consisting of colorectal cancer, prostate cancer, ovarian cancer, non-small cell lung cancer and hepatocellular carcinoma. The presence of a VICKZ expression product in a biological specimen obtained from the individual is an indication that the individual is suffering from metastatic disease, preferably a disease selected from the group consisting of colorectal cancer, prostate cancer, ovarian cancer, non-small cell lung cancer and hepatocellular carcinoma cancer.

Accordingly, in one embodiment the present invention provides a method for the diagnosis of metastatic disease in a subject, the method comprising the steps of:
a) contacting a suitable biological specimen from the subject with a detecting molecule having specific affinity for a VICKZ expression product; and
b) detecting whether the molecule binds to the specimen;

wherein detection of binding between the specimen and the molecule indicates a positive diagnosis of metastatic disease.

Metastatic disease includes a tumor having metastatic potential as well as tumor metastases,per se.

The expression product can be a protein or fragment thereof, in which case the detecting molecule includes an antibody a having a specific affinity for at least one member of the VICKZ family of proteins.

Alternatively, the expression product is an RNA molecule, and in such a case the detecting molecule includes a nucleic acid probe having a sequence complementary to at least a part of the RNA or a cDNA molecule transcribed therefrom.

Antibody is meant to include a molecule comprising the antigen-binding portion of an antibody having specific affinity for at least one VICKZ protein. According to certain embodiments the antibody is selected from the group consisting of a polyclonal antibody, a monoclonal antibody, a proteolytic fragment of an antibody, a chimeric antibody and a recombinant antibody. Recombinant and engineered antibodies, and fragments thereof, include single chain antibodies including single chain composite polypeptides having antigen binding capabilities and comprising amino acid sequences homologous or analogous to the variable regions of an immunoglobulin light and heavy chain i.e. linked v_{H}-V_{L} or single chain Fv (scFv).

According to one embodiment the antibody having a specific affinity for VICKZ is a polyclonal antibody. According to one preferred embodiment the polyclonal antibody is an anti-pan-VICKZ. According to another preferred embodiment the anti-pan-VICKZ is an antibody generated against the Xenopus VGIRBP polypeptide variant D set forth in SEQ ID NO:1. Preferably the anti-pan-VICKZ antibody is an affinity purified antibody.

According to yet another embodiment the antibody having a specific affinity for VICKZ is an antibody generated against a VICKZ peptide.

In one embodiment the VICKZ peptide antibody binds to a human VICKZ1 peptide having the amino acid sequence 5′ GCHQKGQSNQAQA, set forth in SEQ ID NO:2. In another embodiment the VICKZ peptide antibody binds to a human VICKZ2 peptide having the amino acid sequence 5′ GCEQKYPQGVASQRSK, set forth in SEQ ID NO:3. In yet another embodiment the VICKZ peptide antibody binds to a human VICKZ3 peptide having the amino acid sequence 5′ GCQKALQSGPPQS, set forth in SEQ ID NO:4.

Other diagnostic methods include the detection of a VICKZ transcriptional product in a biological sample obtained from the subject comprising contacting said sample with a VICKZ specific nucleic acid probe. In one preferred embodiment the nucleic acid probe is selected from the group consisting of VICKZ RNA or a fragment thereof, VICKZ cDNA or a fragment thereof and a VICKZ-specific oligonucleotide primer.

According to one embodiment the nucleic acid probe is selected from VICKZ cDNA and a fragment of VICKZ cDNA.

For reference purposes, the sequence of the full length xVICKZ3 mRNA polynucleotide is set forth in SEQ ID NO:5.

According to one embodiment a suitable biological specimen or sample from a subject is a bodily fluid or tissue sample from the subject, the subject having or suspected of having cancer. A suitable biological specimen includes, but is not limited to, colorectal tissue or cells, blood serum, lymph node tissue or cells, spleen, liver or lung tissue or cells, ascitic fluid obtained from the abdominal cavity, fecal material and fluid or phlegm obtained from the lung. In one preferred embodiment, the suitable biological specimen is lymph node tissue. In another preferred embodiment the suitable biological specimen is colorectal tissue. Alternatively, the biological specimen may be cells or tissue isolated from the subject that have been cultured in cell culture. Methods of obtaining a suitable biological specimen from a subject are known to those skilled in the art.

In another aspect the present invention provides a kit for the diagnosis of a disorder selected from the group consisting of lymphoma and metastatic disease, the kit comprising:
a) at least one type of binding molecule specific for a VICKZ expression product;
b) means for detecting whether the specific binding molecule is bound to said VICKZ expression product.

In one embodiment the present invention provides a kit for the differential diagnosis of B cell lymphoma subtypes. In another embodiment the present invention provides a kit for the diagnosis of the metastatic disease in a subject.

The expression product can be a protein, in which case the binding molecule includes an antibody having specific affinity for at least one member of the VICKZ family of proteins. The means include an anti-VICKZ antibody comprising a detectable label or a secondary antibody having affinity to the anti-VICKZ antibody, which secondary antibody comprises a detectable label.

Alternatively, the VICKZ expression product is a VICKZ gene transcript, an RNA molecule and in such a case kit comprises at least one nucleic acid molecule having a sequence complementary to at least a part of the VICKZ expression product. The nucleic acid molecule (probe) may be detected by a molecule having affinity for said probe or preferably may be a labelled probe. In another embodiment the kit may comprise primers and optionally reagents for the amplification of the nucleic acid expression product.

In yet another aspect the present invention provides immunogenic and therapeutic compositions and strategies for treating cancers, including therapies aimed at inhibiting the transcription, translation, processing or function (activity) of VICKZ as well as cancer vaccines. In some embodiments the cancer is selected from colorectal cancer, prostate cancer, ovarian cancer, non-small cell lung cancer, hepatocellular cancer and lymphoma. In certain embodiments lymphoma is germinal center B cell derived lymphoma.

In one embodiment the present invention provides a pharmaceutical composition useful for the prevention, attenuation or treatment of a disorder selected from the group consisting of germinal center B cell derived lymphoma and metastatic disease, the composition comprising a VICKZ inhibitor, and a pharmaceutically acceptable diluent or exicipient. In some embodiments metastatic disease is selected from ovarian cancer, colorectal cancer, prostate cancer, ovarian cancer, non-small cell lung cancer and hepatocellular carcinoma.

The present invention further provides the use of a VICKZ specific inhibitor for the preparation of a medicament for preventing and treating lymphoma, germinal center B cell derived lymphoma and metastatic disease.

According to one embodiment the VICKZ inhibitor is a VICKZ specific RNA interference (RNA) molecule. According to one preferred embodiment the RNAi molecule is selected from dsRNA (double stranded RNA)₅ siRNA (small inhibiting RNA), antisense RNA, micro RNA and a ribozyme. In another embodiment the VICKZ specific inhibitor is triple-helix DNA.

In other embodiments the VICKZ inhibitor is a peptide inhibitor selected from a peptide analog having an amino acid sequence derived from the VICKZ polypeptide sequence. The peptide analog is selected from the group consisting of a linear peptide, a cyclic peptide and a peptidomimetic, preferably based on the structure of a VICKZ peptide. In some embodiments the peptide or peptidomimetic is derivatized, linked to a moiety or encapsulated in a vehicle enabling its penetration through the cell membrane.

In some embodiments the inhibitor is a dominant negative protein. In one embodiment the dominant negative protein is a VICKZ protein, which lacks most of the KH4 domain but retains a putative C-terminal dimerization domain. In one preferred embodiment the inhibitor is a dominant negative protein having an amino acid sequence set forth in SEQ ID NO:15, encoded by a DNA sequence set forth in SEQ ID NO:14.

In other embodiments the inhibitor is an anti-VICKZ antibody that is derivatized, linked to a cell-penetrating moiety or encapsulated in a vehicle enabling its penetration through the cell membrane. Non-limiting examples of a cell penetrating moiety or encapsulating vehicle include TAT protein and a lipophilic carrier.

In some embodiments the present invention provides an anti-cancer vaccine. The invention provides an anti-cancer vaccine comprising a VICKZ polypeptide or fragment thereof.

In yet another aspect the present invention provides a method for preventing, attenuating or treating a disorder selected from lymphoma and metastatic disease comprising administering to a subject in need thereof a therapeutically effective amount of at least one VICKZ specific inhibitor and a pharmaceutically acceptable carrier. In some embodiments the lymphoma is GC B-cell specific lymphoma.

The present invention further provides a method of generating a mammalian immune response directed to a VICKZ protein, the method comprising the step of:
a) exposing cells of the mammal's immune system to a molecule selected from a VICKZ polypeptide or immunogenic fragment thereof and a nucleotide sequence that encodes said protein or immunogenic fragment thereof;
whereby an immune response is generated to said protein.

### BRIEF DESCRIPTION OF THE FIGURES

FIG. 1 shows a western blot of VICKZ transformed HEK-293 cells probed with the pan-VICKZ antibody.

FIG. 2 shows VICKZ immunohistological staining of centroblasts and centrocytes, the two types of B cells present in Germinal Centers of B-cells in diseased and healthy tissue.

FIG. 3 shows a graph representing the level of expression of the VICKZ proteins in the lymph node metastases compared to that in normal surrounding colonic epithelium, adenomas, or adenocarcinomas, with statistical analysis one-way ANOVA using a Kruskal-Wallis test.

FIG. 4 depicts VICKZ expression as a predictor of CRC lymph node metastasis. Samples representing invasive disease with a single lesion and with no distal metastases and either no lymph node involvement (T₃N₀) or greater than 3 lymph node metastases (T₃N₂), were stained with the pan VICKZ antibody. FIGS. 4A-4D show representative samples of the scoring scale used for this series, scored 0-3, respectively. FIG. 4E shows a graph of the mean score and standard error of mean for pan-VICKZ expression in the invasive CRC cohort (Cohort #2). A significant correlation between metastasis and hVICKZ level of expression was observed.

FIG. 5 shows anti-pan VICKZ antibody immunohistochemically stained tissue. FIG. 5A shows staining of moderately differentiated adenocarcinoma of the colon (dark arrow adenocarcinomas) while the normal epithelia and surrounding tissue are completely negative (white arrow). FIG. 5B shows staining of a lymph node metastasis from a moderately differentiated adenocarcinoma. Metastases stain strongly for VICKZ protein expression (white arrows), while the adjacent normal lymphoid tissue does not express the proteins, with the notable exception of the germinal centers (black arrows).

FIG. 6A shows immunocytocriemical staining of VICKZ proteins colocalizing with β-actin mRNA to the leading edge in SW480 cell induced to express exogenous VICKZ (uninduced upper row, induced lower row). FIG. 6B shows time-lapse microscopy of SW480 cells transfected with GFP-hVICKZ1 and their protruding lamellae.

FIGS. 7A and 7B show the results of the dominant negative protein.

### DETAILED DESCRIPTION OF THE INVENTION

One aspect of the present invention is based on the unexpected discovery that VICKZ proteins are expressed in the Germinal Centers (GC) of normal, adult lymph nodes. GCs in the lymph nodes represent foci in which B-cells undergo somatic hypermutation and clonally expand when presented with a matching antigen. Centroblasts and centrocytes, the two types of B cells present in GCs, express high levels of VICKZ proteins in their cytoplasm. This specificity enables use of an antibody against VICKZ in the diagnosis of lymphoma, preferably in the differential diagnosis of lymphoma, specifically in diagnosing B-cell lymphomas having a GC origin.

Differential diagnosis (DDs), for example in cases of Hodgkin vs. anaplastic large T-cell lymphomas, or in follicular vs. marginal zone lymphomas of the skin, is often difficult to make using the available set of antibodies used in lymphoma diagnosis. For example, proper staging of FL is dependent on assessing the ratio of the two types of B-cells found in GCs, centroblasts and centrocytes and since the available antibody that recognizes GC-derived B-cells (anti-Bcl-6) stains nuclei, distinction between the two cell types is difficult to make. VICKZ expression is cytoplasmic thus detection of VICKZ expression provides a valuable and straightforward diagnostic tool for hematopathologists.

Additionally, recent evidence suggests that DLBCL, a large, diverse class of B-cell lymphomas, may be classified in two distinct subgroups, one of GC origin and the other derived from activated B-cells (ABC) (Alizadeh et al., 2000). These subgroups appear to have very different prognoses following chemotherapy (Rosenwald et al., 2002), and there is no straightforward method for classifying a given DLBCL as belonging to one or the other subgroup. Anti-VICKZ staining in these cases may prove to be important not only for distinguishing between these two subgroups but also for indicating their prognosis following therapy.

Furthermore, the present invention provides methods useful for diagnosing the metastatic potential of a tumor and compositions and methods useful for treating metastatic disease. The present invention is based on the unexpected discovery that VICKZ proteins are strongly expressed in lymph node and liver metastases o f colorectal carcinomas (CRC). It is now disclosed for the first time that VICKZ proteins are expressed in more than 60% of colorectal carcinomas in a gradient of expression: low to medium levels are detected in moderately to highly dysplastic tubular adenomas, higher levels in metaplastic epithelia, and strong expression in lymph node and liver metastases.

Furthermore, this is the first disclosure of a direct correlation between VICKZ protein expression and the metastatic potential of a cancer, and enables diagnosis of metastatic disease by detecting at least one VICKZ polypeptide or polynucleotide in a biological specimen. The art has neither taught the detection of VICKZ expression in a tumor to identify its metastatic potential nor methods of treating metastatic disease using VICKZ modulators.

An antibody raised to VgI RBP, a Xenopus VICKZ3 protein, was shown to cross react with VICKZ proteins from several vertebrate species, including mouse, chick and all three known human isoforms. This antibody, referred to herein as anti-pan-VICKZ, was further shown to specifically label germinal center (GC) B-cells and B-cell derived lymphomas, including follicular lymphoma (FL) of all grades, diffuse large B-cell lymphoma (DLBCL) and Hodgkin's lymphoma (HL). This antibody provides an excellent tool for the differential diagnosis of lymphoma subtypes, specifically those subtypes derived from GC B cells.

### DEFINITIONS

For convenience certain terms employed in the specification, examples and claims are described herein.

The term “VICKZ” as used herein and in the claims refers to highly conserved family of RNA binding proteins (RBP) and the corresponding polynucleotides. “VICKZ” is an acronym of the first letters of the founding members of this family (VgI RBP, IMP (IGF-II mRNA-binding protein), CRD-BP (c-myc coding region determinant binding protein), KOC (KH-domain containing protein over expressed in cancer), ZBP-I (zipcode binding protein)). The known vertebrate members of this family of proteins each have two N-terminal RNA recognition motifs, an RGG RNA binding domain and four C-terminal hnRNP K-homology (KH) domains (Yaniv and Yisraeli, 2002).

The term “expression product” is used herein to denote a VICKZ protein, or a fragment thereof, a VICKZ RNA, especially, mRNA or fragment thereof as well as cDNA reverse transcribed from said RNA. A VICKZ expression product can be an a transcription or a translation product, or a fragment of a transcription or a translation product from any one or more of the VICKZ isoforms.

“Nucleic acid molecule”, “nucleic acid sequence” or “polynucleotide” as used herein refer to an oligonucleotide, polynucleotide or nucleotide and fragments or portions thereof, and to DNA or RNA of genomic or synthetic origin, which may be single- or double-stranded, and represent the sense or antisense strand.

Throughout the specification and the claims that follow, the term “VICKZ specific” refers to any modulator that has higher affinity or binding to at least one member of the VICKZ family of proteins or fragments thereof or to the nucleic acids encoding same, than to another protein or nucleic acid.

The term “metastatic disease” includes a tumor having metastatic potential as well as tumor metastases, per se. Specifically metastatic disease refers to cancers having a metastatic potential and to metastases that have spread to regional lymph nodes or to distant sites. In preferred embodiments of the present invention metastatic disease refers to colorectal cancer, prostate cancer, ovarian cancer, non-small cell lung cancer and hepatocellular cancer and the metastases derived therefrom. The most common sites for colon cancer metastasis, for example, are lymph node, lung, bone and liver.

According to certain embodiments a suitable biological specimen from a subject is a bodily fluid or tissue from the subject, which contains lymphoma cells. A suitable biological sample includes, but is not limited to, serum, lymph node and bone marrow. In a preferred embodiment, the suitable biological sample is lymph node tissue.

According to other embodiments a suitable biological specimen from a subject is a bodily fluid or tissue from the subject, which would contain tumor cells. A suitable biological specimen includes, but is not limited to any histological sample. Certain embodiments include colorectal tissue or cells, serum, lymph node tissue, liver tissue, fecal material and lung biopsies. Other suitable biological specimens include ascites fluid obtained from the abdomen of a patient suspected of having or having ovarian cancer and fluid or phlegm from the lung. In a preferred embodiment, the suitable biological sample is lymph node tissue.

The biological specimen may be cells or tissue from the subject that have been cultured in cell culture. Methods of obtaining a suitable biological sample from a subject are known to those skilled in the art.

In another aspect the present invention provides a kit for the diagnosis of a disorder selected from the group consisting of lymphoma and metastatic disease, the kit comprising a binding molecule specific for a VICKZ sequence selected from the group consisting of a VICKZ specific peptide, a VICKZ specific polypeptide and a VICKZ specific polynucleotide, and means for detecting whether the specific binding molecule is bound to said VICKZ sequence.

In one embodiment the present invention provides a kit for the differential diagnosis of lymphoma subtypes, the kit comprising a binding molecule specific for a VICKZ sequence selected from the group consisting of a VICKZ specific peptide, a VICKZ specific polypeptide and a VICKZ specific polynucleotide, and means for detecting whether the specific binding molecule is bound to said VICKZ sequence.

In another embodiment the present invention provides a kit for the diagnosis of the metastatic potential of a tumor in a subject, the kit comprising a binding molecule specific for a VICKZ sequence selected from the group consisting of a VICKZ specific peptide, a VICKZ specific polypeptide and a VICKZ specific polynucleotide, and a means for detecting whether the specific binding molecule is bound to a said VICKZ sequence.

Antibodies

According to one embodiment the present invention provides a method for the differential diagnosis of lymphoma subtypes wherein the method comprises contacting a suitable biological specimen from the subject with a molecule comprising the antigen-binding portion of an antibody having a specific affinity for VICKZ; and detecting whether the molecule binds to the specimen, wherein detection of binding between the specimen and the molecule provides a positive indication in the diagnosis of GC B-cell derived lymphoma. GC derived B-cell lymphoma includes non-Hodgkin's lymphomas such as follicular lymphomas and diffuse large B cell lymphomas, and the Reed-Sternberg cells in Hodgkin's lymphomas. The molecule comprising the antigen-binding portion of an antibody having a specific affinity for VICKZ may be an antibody such as a polyclonal or monoclonal antibody, or a proteolytic fragment thereof such as an Fab or F(ab′)₂ fragment. Additional embodiments include chimeric antibodies; human and humanized antibodies; recombinant and engineered antibodies, and fragments thereof, including single chain antibodies. Single chain antibodies can be single chain composite polypeptides having antigen binding capabilities and comprising amino acid sequences homologous or analogous to the variable regions of an immunoglobulin light and heavy chain i.e. linked V_{H}-V_{L} or single chain Fv (scFv).

Antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (fragment variable), Fab fragments and Fc (fragment crystalline) domains, depending on the proteolytic enzyme. The antigen binding domains, Fab, include regions where the polypeptide sequence varies. The term F(ab′)₂ represents two Fab′ arms linked together by disulfide bonds. The central axis of the antibody is termed the Fc fragment. Each heavy chain has at one end a variable domain (V_{H}) followed by a number of constant domains (C_{H})—Each light chain has a variable domain (V_{L}) at one end and a constant domain (C_{L}) at its other end, the light chain variable domain being aligned with the variable domain of the heavy chain and the light chain constant domain being aligned with the first constant domain of the heavy chain (CH1).

The variable domains of each pair of light and heavy chains form the antigen-binding site. The domains on the light and heavy chains have the same general structure and each domain comprises four framework regions, whose sequences are relatively conserved, joined by three hypervariable domains known as complementarity determining regions (CDR1-3). These domains contribute specificity and affinity of the antigen-binding site.

The isotype of the heavy chain (gamma, alpha, delta, epsilon or mu) determines immunoglobulin class (IgG, IgA, IgD, IgE or IgM, respectively). The light chain is either of two isotypes (kappa, K or lambda, λ) found in all antibody classes.

Further included within the scope of the invention are chimeric antibodies; human and humanized antibodies; recombinant and engineered antibodies, and fragments thereof. Furthermore, the DNA encoding the variable region of the antibody can be inserted into the DNA encoding other antibodies to produce chimeric antibodies (see, for example, U.S. Pat. No. 4,816,567).

Antibody engineering can join the separate segments of the heavy and light chains in the Fv with a flexible peptide linker to form a single-chain Fv (scFv), the scope of which fall within the present invention. Single chain antibodies can be single chain composite polypeptides having antigen binding capabilities and comprising amino acid sequences homologous or analogous to the variable regions of an immunoglobulin light and heavy chain (linked V_{H}-VL or single chain Fv (ScFv)). Both V_{H} and V_{L} may copy natural monoclonal antibody sequences or one or both of the chains may comprise a CDR-FR construct of the type described in U.S. Pat. No. 5,091,513, the entire contents of which are incorporated herein by reference. The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are incorporated herein by reference.

Additionally, CDR grafting may be performed to alter certain properties of the antibody molecule including affinity or specificity. A non-limiting example of CDR grafting is disclosed in U.S. Pat. No. 5,225,539.

A “molecule having the antigen-binding portion of an antibody” as used herein is intended to include not only intact immunoglobulin molecules of any isotype and generated by any animal cell line or microorganism, but also the antigen-binding reactive fraction thereof, including, but not limited to, the Fab fragment, the Fab¹ fragment, the F(ab′)₂ fragment, the variable portion of the heavy and/or light chains thereof, Fab miniantibodies (see WO 93/15210, U.S. patent application Ser. No. 08/256,790, WO 96/13583, U.S. patent application Ser. No. 08/817,788, WO 96/37621, U.S. patent application Ser. No. 08/999,554, the entire contents of which are incorporated herein by reference), dimeric bispecific miniantibodies (see Muller, et al, 1998) and chimeric or single-chain antibodies incorporating such reactive fraction, as well as any other type of molecule or cell in which such antibody reactive fraction has been physically inserted, such as a chimeric T-cell receptor or a T-cell having such a receptor, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

The term “Fc” as used herein is meant as that portion of an immunoglobulin molecule (Fragment crystallizable) that mediates phagocytosis, triggers inflammation and targets Ig to particular tissues; the Fc portion is also important in complement activation.

The term “epitope” is meant to refer to that portion of any molecule capable of being bound by an antibody or a fragment thereof which can also be recognized by that antibody. Epitopes or antigenic determinants usually consist of chemically active surface groupings of molecules such as amino acids or sugar side chains and have specific three-dimensional structural characteristics as well as specific charge characteristics.

An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

A “neutralizing antibody” as used herein refers to a molecule having an antigen-binding site to a specific receptor capable of reducing or inhibiting (blocking) activity or signaling through a receptor, as determined by in vivo or in vitro assays, as per the specification.

A “monoclonal antibody” or “mAb” is a substantially homogeneous population of antibodies to a specific antigen. mAbs may be obtained by methods known to those skilled in the art. See, for example Kohler et al (1975); U.S. Pat. No. 4,376,1 10; Ausubel et al (1987-1999); Harlow et al (1988); and Colligan et al (1993), the contents of which references are incorporated entirely herein by reference. The mAbs of the present invention may be of any immunoglobulin class including IgG, IgM, IgE, IgA, and any subclass thereof. A hybridoma producing a mAb may be cultivated in vitro or in vivo. High titers of mAbs can be obtained by in vivo production where cells from the subject hybridomas are injected intraperitoneally into pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

Chimeric antibodies are molecules, the different portions of which are derived from different animal species, such as those having a variable region derived from a murine mAb and a human immunoglobulin constant region. Antibodies which have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Harlow et al, 1988; Liu et al, 1987, European Patent Applications 125023, 171496, 173494, 184187, 173494, PCT patent applications WO 86/01533, WO 97/02671, WO 90/07861, WO 92/22653 and U.S. Pat. Nos. 5,693,762, 5,693,761, 5,585,089, 5,530,101 and 5,225,539). These references are hereby incorporated by reference.

In addition to the conventional method of raising antibodies in vivo, antibodies can be generated in vitro using phage display technology. This technology is much faster than conventional antibody production and antibodies can be generated against an enormous number of antigens. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies one can use various methods all based on phage display libraries to generate a large pool of antibodies with different antigen recognition sites. Protocols for bacteriophage library construction and selection of recombinant antibodies are provided in the reference text Current Protocols in Immunology, Colligan et al (Eds.), John Wiley & Sons, Inc. (1992-200O), Chapter 17, Section 17.1.

Detection of antibody binding may be performed by contacting the antibody-antigen complex with a second antibody linked to an enzyme, such as alkaline phosphatase or horseradish peroxidase, or a fluorescent marker, such as FITC or Cy3. Other enzymes or markers may be employed and are well known to one with skill in the art.

The antibody or fragment thereof can be labelled with a radioisotope for in vivo or in vitro detection of VICKZ proteins. In certain embodiments the method for the detection of VICKZ in a biological sample is performed in vitro, in vivo or in situ detection of the presence of VICKZ in a tissue or cell is contemplated in the present invention.

In some embodiments a VICKZ inhibitor is an anti-VICKZ antibody that has been derivatized, linked to a cell-penetrating moiety (such as TAT protein), or encapsulated or impregnated in a vehicle enabling its penetration through the cell membrane.

Nucleic Acids

In one embodiment, the present invention provides a method for the differential diagnosis of lymphoma subtypes or of metastatic disease in a subject, the method comprising the steps of contacting a suitable biological specimen from the subject with a nucleic acid molecule that selectively hybridizes to a VICKZ RNA molecule, specifically a mRNA molecule, in the specimen, followed by detecting the bound nucleic acid molecule. Detection of binding between the specimen and the nucleic acid molecule indicates a positive diagnosis of GC B-cell derived lymphoma or of a tumor having metastatic potential or a tumor metastases. According to certain embodiments of the present invention, VICKZ expression is detected by contacting a suitable biological specimen with an isolated nucleic acid molecule that selectively hybridizes to VICKZ mRNA. VICKZ mRJMA includes full length mRNA or fragments thereof. A nucleic acid molecule that is used in diagnosis may be referred to as a “probe”. The nucleic acid probes are designed t o be substantially complementary to the VICKZ nucleic acids, i.e. the target sequence, such that hybridization occurs between the probes and the target sequence of the present invention. The complementarity need not be perfect; there may be any number of base pair mismatches which will interfere with hybridization between the target sequence and the nucleic acids of the present invention. However, if the number of mutations is so great that no hybridization can occur under even the least stringent of hybridization conditions, the sequence is not a complementary sequence. Thus, by “substantially complementary” herein is meant that the probes are sufficiently complementary to the target sequences to hybridize under normal reaction conditions, particularly high stringency conditions, as outlined herein.

The protein and DNA sequences of human VICKZ1 are provided herein as SEQ ID NO: 6 and SEQ ID NO: 7, respectively. The protein and DNA sequences of human VICKZ2 splice variant 1 are provided herein as SEQ ID NO: 8 and SEQ ID NO: 9, respectively. The protein and DNA sequences of human VICKZ2 splice variant 2 are provided herein as SEQ ID NO: 10 and SEQ ID NO: 11, respectively. The protein and DNA sequences of human VICKZ3 are provided herein as SEQ ID NO: 12 and SEQ ID NO: 13, respectively. The length of trie nucleic acid molecules may vary, but in general, the probes range from about 10 to about 100 bases long, preferably from about 20 to about 80 bases, and more preferably from about 30 to about 50 bases. In some embodiments, much longer nucleic acids, up to hundreds of bases, and full-length antisense RNA or cDNA are used.

Detection of VICKZ expression may be carried out using an isolated nucleic acid molecule of VICKZ. The isolated nucleic acid molecule may be labeled with a detectable marker. The detectable marker may be a radioactive label, a calorimetric, luminescent, or a fluorescent marker. Other detectable markers are known to those skilled in the art. The nucleic acid may further be labeled with an antigen that can be recognized by an antibody

DNA probe molecules may be produced by insertion of a DNA molecule having the full-length or a fragment of the VICKZ locus into suitable vectors, such as plasmids or bacteriophages, using methods well known in the art. Alternatively, probes may be generated chemically from DNA synthesizers.

RNA probes may be generated by inserting the full length or a fragment of the VICKZ locus downstream of a bacteriophage promoter such as T3, T7 or SP6. Large amounts of RNA probe may be produced by incubating the labeled nucleotides with a linearized VICKZ polynucleotide or a fragment thereof, containing an upstream promoter in the presence of the appropriate RNA polymerase.

This invention provides an antisense molecule capable of hybridizing to the VICKZ polynucleotide, preferably to the VICKZ mRNA encoding at least one member of the VICKZ family of proteins. The antisense molecule may be DNA or RNA or variants thereof (i.e. DNA with a protein backbone). Antisense nucleic acids are DNA or RNA molecules that are complementary to at least a portion of a specific mRNA molecule. In the cell, they hybridize to that mRNA, forming a double stranded molecule. Oligonucleotides of about fifteen nucleotides and molecules that hybridize to the AUG initiation codon will be particularly efficient, since they are easy to synthesize and are likely to pose fewer problems than larger molecules upon introduction to cells.

The terms “nucleic acid” and “polynucleotides” refer to molecules including deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “oligonucleotide” refers to a nucleic acid sequence of at least about 6 nucleotides to about 60 nucleotides, preferably about 15 to 30 nucleotides, and more preferably about 20 to 25 nucleotides, which can be used in PCR amplification or a hybridization assay, or a microarray. As used herein, oligonucleotide is substantially equivalent to the terms “amplimers”, “primers”, “oligomers”, and “probes”, as commonly defined in the art.

The term “peptide nucleic acid” (PNA) as used herein refers to nucleic acid “mimics”; the molecule's natural backbone is replaced by a pseudopeptide backbone and only the four-nucleotide bases are retained. The peptide backbone ends in lysine, which confers solubility to the composition. PNAs may be pegylated to extend their lifespan in the cell where they preferentially bind complementary single stranded DNA and RNA and stop transcript elongation (Nielsen, et al., 1993).

As used herein, highly stringent conditions are those, which are tolerant of up to about 5% to about 25% sequence divergence, preferably up to about 5% to about 15%. Without limitation, examples of highly stringent (−10° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those, which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5× Denhardt's reagent, 0.5% SDS, 100 μg/ml denatured, fragmented salmon sperm DNA at an appropriate incubation temperature Ti. See generally Sambrook et al., Molecular Cloning: A Laboratory Manual, 2d edition, Cold Spring Harbor Press (1989)) for suitable high stringency conditions.

Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization, and the duration of the hybridization. The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na⁺. The rate is directly proportional to duplex length and inversely proportional to the degree of mismatching.

Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA:DNA hybrids using the equation of Meinkoth et al (1984), as
Tm=81.5° C.+16.6 (log M)+0.41 (% GC)−0.61 (% form)−500/L
and for DNA:RNA hybrids, as
Tm=79.8° C.+18.5 (log M)+0.58 (% GC)−11.8 (% GC)²−0.56(% form)−820/L
where M, molarity of monovalent cations, 0.01-0.4 M NaCl,
% GC, percentage of G and C nucleotides in DNA, 30%-75%,
% form, percentage formamide in hybridization solution, and
L, length hybrid in base pairs.

Tm is reduced by 0.5-1.5° C. (an average of 1° C. can be ULsed for ease of calculation) for each 1% mismatching. The Tm may also be determined experimentally. As increasing length of the hybrid (L) in the above equations increases the Tm and enhances stability, the full-length rat gene sequence can be used as the probe.

Filter hybridization is typically carried out at 68° C., and at high ionic strength (e.g., 5-6×SSC), which is non-stringent, and followed by one or more washes of increasing stringency, the last one being of the ultimately desired high stringency. The equations for Tm can be used to estimate the appropriate Ti for the final wash, or the Tm of the perfect duplex can be determined experimentally and Ti then adjusted accordingly.

The invention also provides for conservative amino acid variants of the molecules. Variants according to the invention also may be made that conserve the overall molecular structure of the encoded proteins. Given the properties of the subject amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved.

RNAi

The present invention further provides a method for treating or preventing GC B-cell derived lymphoma and metastatic disease comprising administering to a subject in need thereof a therapeutically effective amount of at least one VICKZ specific inhibitor and pharmaceutically acceptable carrier. VICKZ is a cytoplasmic protein and therefore the VICKZ inhibitor is preferably selected from a RNA interference (RNAi) molecule, a peptide inhibitor and a peptidomimetic. A VICKZ RNAi molecule may be selected from VICKZ specific dsRNA (double stranded RNA), VICKZ specific siRNA (small inhibiting RNA), VICKZ specific antisense RNA, VICKZ specific micro RNA and a VICKZ specific ribozyme.

A preferred peptide inhibitor is a cyclic peptide of the VICKZ protein. In another embodiment the VICKZ specific inhibitor is triple-helix DNA. Without wishing to be bound to theory, triple-helix DNA targets the double-helix DNA strand itself and are designed to bind with a specific section of the DNA, preventing its transcription into RNA. Triple-helix oligonucleotides may serve the same purpose as classical antisense or other RNAi molecules.

VICKZ is a cytoplasmic protein, therefore pharmaceutical compositions that inhibit VICKZ expression or function within the cell are preferred. The present invention also contemplates pharmaceutical formulations, both for veterinary and for human medical use, which comprise as the active agent one or more of the VICKZ inhibitors described in the invention, for the manufacture of a medicament for the treatment or prophylaxis of the conditions variously described herein.

Selective disruption of VICKZ expression is an effective method of treating patients with certain subtypes of lymphoma. CD20, expressed in both normal and neoplastic cells, is a validated target for the treatment of lymphoma and the commercially available anti-CD20 antibody, Rituximab® or Rituxan®, is an effective treatment for patients with non-Hodgkin's lymphoma.

Selection of RNAi sequences for the effective inhibition of RNA is well known to one skilled in the art. For example, guidelines for the selection of highly effective siRNA sequences for mammalian RNA interference are described in Ui-Tei et al. (Ui-Tei et al., 2004). Liao et al (Liao et al, 2004) has described the “knock-out”, or silencing, of the CRD-BP gene in human K562 leukemia cells using the siRNA technology. Lentivirus is a useful vector for the expression and delivery of RNAi (Stewart, et al, 2003)

The ability of a RNA interference molecule containing a given target sequence to cause RNAi-mediated degradation of the target mRNA can be evaluated using standard techniques for measuring the levels of RNA or protein in cells. For example, siRNA of the invention can be delivered to cultured cells, and the levels of target mRNA can be measured by Northern blot or dot blotting techniques, or by quantitative RT-PCR. Alternatively, the levels of VICKZ protein in the cultured cells can be measured by ELISA or Western blot.

Classical RNAi compounds target specific strands of RNA within the cell to bind with, thus preventing the production of that RNA's protein. Degradation of the target mRNA by a VICKZ specific RNAi molecule will reduce the production of a functional gene product from the VICKZ genes. Thus, the invention provides a method of inhibiting expression of the VICKZ proteins in a subject, comprising administering an effective amount of an RNAi molecule of the invention to the subject, such that the target mRNA is degraded.

One skilled in the art can readily determine an effective amount of the RNAi molecules of the invention to be administered to a given subject, by taking into account factors including the size and weight of the subject; the age, health and sex of the subject and the route of administration. Generally, an effective amount of the RNAi of the invention comprises an intercellular concentration from about 1 nanomolar (nM) to about 100 nM, preferably from about 2 nM to about 50 nM, more preferably from about 2.5 nM to about 10 nM. It is contemplated that greater or lesser amounts of RNAi molecules may be administered.

Peptide Analogs

The present invention provides peptide analogs for the inhibition of VICKZ activity. The peptide analogs include linear and cyclic peptides and peptidomimetics. A peptide mimetic or peptidomimetic is a molecule that mimics the biological activity of a peptide but is not completely peptidic in nature. Whether completely or partially non-peptide, peptidomimetics according to this invention provide a spatial arrangement of chemical moieties that closely resembles the three-dimensional arrangement of groups in the peptide on which the peptidomimetic is based. As a result of this similar active-site geometry, the peptidomimetic has effects on biological systems, which are similar to the biological activity of the peptide.

Without wishing to be bound by theory, the present invention encompasses peptide and peptide analog compositions. Said peptide/peptidomimetic compositions are effective in situations where down regulation of VICKZ is desired, particularly in GC B cell lymphomas and metastatic disease of colorectal tissue. In certain embodiment a dominant negative protein is preferred.

There are clear advantages for using a mimetic of a given peptide rather than the peptide itself, because peptides commonly exhibit two undesirable properties: poor bioavailability and short duration of action. Peptide mimetics offer a route around these two major obstacles, since the molecules concerned are have a long duration of action. Small peptidomimetics of 3-6 amino acids exhibit improved patient compliance since they can be administered orally compared with parenteral administration for peptides or larger peptidomimetics. Furthermore there are problems associated with stability, storage and immunoreactivity for peptides that are not experienced with peptide mimetics.

One aspect of the present invention provides for a peptidomimetic or a peptide or peptide analog, which mimics the structural features of the critical minimal epitope.

The design of the peptidomimetics may be based on the three-dimensional structure of VICKZ with or in complex with RNA. Binding of the peptidomimetic either induces the binding protein to carry out the normal function caused by such binding (agonist) or disrupts such function (antagonist, inhibitor).

A primary goal in the design of peptide mimetics has been to reduce the susceptibility of mimics to cleavage and inactivation by peptidases. In one approach, one or more amide bonds have been replaced in an essentially isosteric manner by a variety of chemical functional groups. In another approach, a variety of uncoded or modified amino acids such as D-amino acids and N-methyl amino acids have been used to modify mammalian peptides. Alternatively, a presumed bioactive conformation has been stabilized by a covalent modification, such as cyclization or by incorporation of γ-lactam or other types of bridges as disclosed for example in U.S. Pat. No. 5,811,392. In U.S. Pat. No. 5,552,534, non-peptide compounds are disclosed which mimic or inhibit the chemical and/or biological activity of a variety of peptides. Such compounds can be produced by appending to certain core species, such as the tetrahydropyranyl ring, chemical functional groups, which cause the compounds to be at least partially cross-reactive with the peptide. As will be recognized, compounds which mimic or inhibit peptides are to varying degrees cross-reactive therewith. Other techniques for preparing peptidomimetics are disclosed in U.S. Pat. No. 5,550,251 and U.S. Pat. No. 5,288,707, for example. Non-limiting examples of the use of peptidomimetics in the art include inhibitors of protein isoprenyl transferases (particularly protein farnesyltransferase and geranylgeranyltransferase) and anti-cancer drugs (U.S. Pat. No. 5,965,539) inhibitors of p21 ras (U.S. Pat. No. 5,910,478 ) and inhibitors of neurotropin activity (U.S. Pat. No. 6,291,247).

VICKZ Anti-Cancer Vaccines

The specificity of VICKZ expression in cancers makes them a good target for anti-cancer vaccines. Accordingly the present invention provides cancer vaccines comprising a VICKZ-related protein or VICKZ-related nucleic acid hi view of the expression pattern of VICKZ, anti-cancer vaccines prevent and/or treat VICKZ-expressing cancers with minimal or no effects on non-target tissues. The use of a tumor antigen in a vaccine that generates humoral and/or cell-mediated immune responses as anti-cancer therapy is known in the art (reviewed in e.g., Henderson, et al, 2005; Knutson and Disis, 2005; Timmerman and Levy, 2000).

An anti-cancer vaccine is prepared by employing a VICKZ-related protein, or a VICKZ-encoding nucleic acid molecule and recombinant vectors capable of expressing and presenting the VICKZ immunogen. Skilled artisans understand that a wide variety of vaccine systems for delivery of immunoreactive epitopes are known in the art including antigen loaded dendritic cells, recombinant viral vectors, liposomes and the like. Briefly, such methods of generating an immune response (e.g. humoral and/or cell-mediated) in a mammal comprise the steps of: exposing the mammal's immune system to an immunoreactive epitope so that the mammal generates an immune response that is specific for that epitope. Therefore, a mixture of the VICKZ protein, a single VICKZ protein, immunogenic regions or epitopes thereof can be combined and delivered by various means.

In patients with VICKZ-associated lymphoma or metastatic disease, the vaccine compositions of the invention can also be used in conjunction with other treatments used for cancer, e.g., surgery, chemotherapy, drug therapies and radiation therapies.

Cytotoxic T-cell (CTL) epitopes can be determined using specific algorithms to identify peptides within VICKZ protein that bind corresponding HLA alleles Generally HLA Class I epitopes are about 8 to about 12 amino acids long. In contrast, the HLA Class II epitopes are about 9 or more amino acids. Antibody-based Vaccines

A wide variety of methods for generating an immune response in a mammal are known in the art (for example as the first step in the generation of hybridomas). Methods of generating an immune response in a mammal comprise exposing the mammal's immune system to an immunogenic epitope on a protein so that an immune response is generated. A typical embodiment consists of a method for generating an immune response to VICKZ in a host, by contacting the host with a sufficient amount of at least one VICKZ B cell or T-cell epitope or analog thereof; and at least one periodic interval thereafter re-contacting the host with the VICKZ B cell or T-cell epitope or analog thereof. An immune response can also be elicited by exposing a mammal to a multiepitopic peptide. Typically, such vaccine preparations further contain a suitable adjuvant.

Accordingly, the present invention provides a composition comprising a VICKZ protein comprising at least one T cell or at least one B cell epitope. Without wishing to be bound to theory, upon contact of the epitope with a mammalian immune system T cell or B cell respectively, the T cell or B cell is activated. When the immune system cell is a B cell, the activated B cell generates antibodies that specifically bind to the VICKZ protein. When the immune system cell is a T cell that is a cytotoxic T cell (CTL), the activated CTL kills an autologous cell that expresses the VICKZ protein. When the immune system cell is a T cell that is a helper T cell (HTL), the activated HTL secretes cytokines that facilitate the cytotoxic activity of a cytotoxic T cell (CTL) or the antibody-producing activity of a B cell.

Vaccine compositions of the invention include nucleic acid-mediated modalities. DNA or RNA that encode VICKZ protein(s) of the invention can be administered to a patient. Genetic immunization methods can be employed to generate prophylactic or therapeutic humoral and cellular immune responses directed against cells expressing VICKZ. Constructs comprising DNA encoding a VICKZ-related protein/immunogen and appropriate regulatory sequences can be injected directly into muscle or skin of an individual, such that the cells of the muscle or skin take-up the construct and express the encoded VICKZ protein/immunogen. Alternatively, a vaccine comprises a VICKZ-related protein.

For therapeutic or prophylactic immunization purposes, proteins of the invention can be expressed via viral or bacterial vectors. Various viral gene delivery systems that can be used in the practice of the invention are known in the art and include, but are not limited to, vaccinia, fowlpox, canarypox, adenovirus, influenza, poliovirus, adeno-associated virus, lentivirus and sindbis virus. Thus, gene delivery systems are used to deliver a VICKZ-related nucleic acid molecule. In one embodiment, full-length VICKZ cDNA is employed. In another embodiment, VICKZ nucleic acid molecules encoding specific cytotoxic T lymphocyte (CTL) and/or antibody epitopes are employed. VICKZ refers to VICKZ1, VICKZ2 and or VICKZ3.

Various ex vivo strategies can also be employed to generate an immune response. One approach involves the use of antigen presenting cells (APCs) such as dendritic cells (DC) to present VICKZ antigen to a patient's immune system. In one embodiment, autologous dendritic cells are pulsed with VICKZ peptides capable of binding to MHC class I and/or class II molecules. In another embodiment, dendritic cells are pulsed with the complete VICKZ protein. Yet another embodiment involves engineering the expression of the VICKZ gene in dendritic cells using various implementing vectors known in the art, such as adenovirus, retrovirus, lentivirus, adeno-associated virus, DNA transfection, or tumor-derived RNA transfection. Accordingly, the present invention provides a pharmaceutical composition comprising: (a) an antigen presenting cell that expresses a VICKZ polypeptide or peptide and (b) a pharmaceutically acceptable carrier or excipient.

Anti-idiotypic anti-VICKZ antibodies can also be used in anti-cancer therapy as a vaccine for inducing an immune response to cells expressing a VICKZ -related protein. In particular, the generation of anti-idiotypic antibodies is well known in the art; this methodology can readily be adapted to generate anti-idiotypic anti VICKZ antibodies.

Pharmaceutical Compositions

The present invention provides pharmaceutical compositions comprising a polypeptide or nucleic acid as described above and a physiologically acceptable carrier.

In some embodiments the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

In other embodiments the present invention provides pharmaceutical compositions comprising: (a) an antigen presenting cell that expresses a VICKZ polypeptide or peptide and (b) a pharmaceutically acceptable carrier or excipient. Illustrative antigen presenting cells include dendritic cells, macrophages, monocytes, fibroblasts and B cells. Additionally, pharmaceutical compositions are provided that comprise: (a) an antigen presenting cell that expresses a VICKZ polypeptide or a peptide and (b) an immunostimulant.

In pharmaceutical and medicament formulations, the active agent is preferably administered together with one or more pharmaceutically acceptable carrier(s) and optionally any other therapeutic agents. The active agent is provided in an amount effective to achieve the desired pharmacological effect, as described above, and in a quantity appropriate to achieve the desired daily dose. Therapeutic molecules of the present invention include RNAi molecules, peptides, peptide analogs, derivatized antibodies and anti-VICKZ vaccines.

For treating lymphoma, the RNAi molecules of the invention can be administered to a subject in combination with a therapeutic agent, different from the present RNAi. For example, the RNAi of the invention can be administered in combination with therapeutic methods currently employed for treating cancer or preventing tumor metastasis (e.g., radiation therapy, chemotherapy, and surgery). For treating lymphoma, the RNAi of the invention is preferably administered to a subject in combination with radiation therapy, or in combination with chemotherapeutic agents such as anti-CD20 or anti-bcl-6 antibody.

In the present methods, the present RNAi can be administered to the subject in need thereof either as naked RNAi, in conjunction with a delivery reagent, or as a recombinant plasmid or viral vector, which expresses the RNAi molecule. Suitable delivery reagents for administration in conjunction with the present RNAi include liposomes. Liposomes suitable for use in the invention are formed from standard vesicle-forming lipids, which generally include neutral or negatively charged phospholipids and a sterol, such as cholesterol. The selection of lipids is typically guided by consideration of factors such as the desired liposome size and half-life of the liposomes in the blood stream. The liposomes encapsulating the RNAi of the present invention may further comprise a targeting moiety useful in targeting the liposome to a particular cell or tissue.

According to certain embodiments the VICKZ inhibitor is a peptide inhibitor selected from peptide analogs having amino acid sequence derived from the VICKZ polypeptide sequence and peptidomimetics based on the structure of such peptides.

Without wishing to be bound to theory the peptide inhibitor is designed to interfere with VICKZ protein-protein or VICKZ protein-RNA interactions. A non-limiting example of a peptide designed to interfere with protein-protein interactions is SAHB (stabilized a helix of BCL2 domains). SAHB is a helical, protease resistant, cell permeable peptidomimetic useful for activation of apoptosis in cancer cells (Walensky et al., 2004). Another example is a peptidomimetic designed to mimic the protein-protein interactions of an apoptotic activator, SMAC (Li et al, 2004). Other examples of peptide inhibitors include small peptides, such as a tetrapeptide that preferentially blocks the polymerization of a pathologically unstable seipin commonly present in Alzheimer patients of European decent (Zhou, et al., 2004).

The pharmaceutical composition of this invention may be administered by any suitable means, such as orally, topically, intranasally, subcutaneously, intramuscularly, intravenously, intra-arterially, intraarticularly, intralesionally or parenterally. Ordinarily, intravenous (i.v.), intraarticular, oral or parenteral administration will be preferred.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician. As used herein, a “therapeutically effective amount” refers to the amount of a molecule required to alleviate one or more symptoms associated with a disorder being treated over a period of time.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those in the art. (See, for example, Ansel et al., 1990 and Gennaro, 1990). In addition, if desired, the composition can contain minor amounts of auxiliary substances such as wetting or emulsifying agents, pH buffering agents.

The present invention further provides the use of a VICKZ inhibitor for the preparation of a medicament for the treatment of a disorder selected from GC B cell derived lymphomas or metastatic disease.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein. Additionally, the entire contents of the references cited within the references cited herein are also entirely incorporated by references. The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

### Example 1

### Antibody Preparation

The anti-pan VICKZ antibody was raised by inoculating rabbits with a histidine-tagged Xenopus VICKZ fusion protein. The fusion protein was prepared according to methods known in the art, using the Xenopus VICKZ3 cDNA set forth as SEQ ID NO:5

The His-tagged full-length xVICKZ3 (VgI-RBP) recombinant protein was prepared as previously described using a pET21d expression vector system (Zhang, 1999). After purification using nickel column chromatography following manufacturer's protocol (QIAGEN Inc., Valencia, Calif.), an aliquot of the purified protein batch was subjected to SDS-polyacrylamide gel electrophoresis. The gel was stained by coomasle blue to confirm the recombinant xVICKZ3 protein purity and length. Rabbit anti-xVICKZ3 serum was raised by immunization with the recombinant full-length xVICKZ3, following standard protocol. The affinity of the antisera was tested by western blot analysis. Antisera were purified using xVICKX3 protein column chromatography. The polyclonal anti-VICKZ3 antibodies were released from the column using 0.1M glycine pH 4, and immediately titrated with 1M Tris pH 7.4. BSA was added to reach a final concentration of 1%. Dialysis against IXPBS was performed and sodium azide was added. Frozen aliquots were thawed before use. The purified antisexa recognized all three human VICKZ isoforms, hence the label “anti-pan VICKZ” antibody.

### Example 2

### Western Blot Analysis

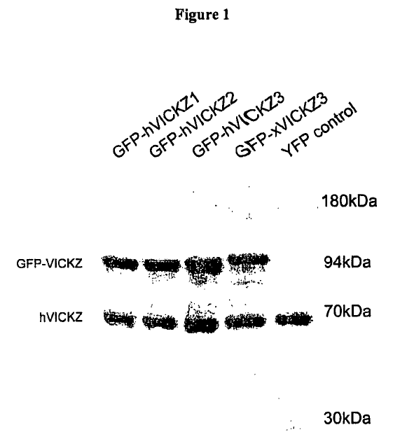

HEK-293 cells were transfected with GFP-hVICKZ1 (human VICKZ1), GFP-hVICKZ2, GFP-hVICKZ3, GFP-xVICKZ3 and YFP-Cl as a control. Total cell extracts from these cells were subjected to western blot analysis, using the pan-VICKZ antibody. FIG. 1 shows the western blot with protein marker sizes indicated. Two specific bands are observed; the 94 kDa band represents the GFP chimeric hVICKZ proteins (GFP-xVICKZ3 is approximately 97 kDa). The endogenous hVICKZ proteins are also recognized and were used as loading controls. The high specificity of the antibody is evident from the absence of other detectable bands. All lanes have similar signal intensity, showing equal affinity to the members of the VICKZ protein family, regardlesss of species or isoform.

### Example 3

### Specific Anti-VICKZ Antibodies

Anti-VICKZ peptide antibodies to three different epitopes of the human VICKZ1, VICKZ1 and VICKZ 3 proteins were generated. The peptides GCHQKGQSNQAQA (corresponding to amino acids 564-574 of VICKZ1, SEQ ID NO: 2) and GCEQKYPQGVASQRSK (amino acids 585-598 of VICKZ2; SEQ ID NO: 3) and GCQKALQSGPPQS (amino acids 566-576 of human VICKZ3; SEQ ID NO:4) were each synthesized and injected into rabbits. The resulting polyclonal sera were used to probe western blots and histological sections from patients with various malignant diseases.

### Example 4

### VICKZ-Specific Nucleic Acid Probes and Primers

VICKZ specific nucleic acid probes are useful for detecting VICKZ RNA in cells or tissue samples. The sequences may be DNA or RNA and may be elected using standard computer algorithms, known to those with skill in the art.

### Example 5

### VICKZ Immunostaining

Formadehyde-fixed, paraffin-embedded sections were deparaffinized, and antigen retrieval performed using 0.1M glycine pH 9. Following hydrogen peroxide treatment, samples were incubated with purified pan-VICKZ antibody at a titer of 1:800 overnight at 4 degrees C. After washes with OPTIMAX®, anti-rabbit HRP-conjugated ENVISION® antibody was added for an hour at RT. Following washes, slides were developed using AEC for 15 minutes or DAB for 7 minutes, washed, and stained with hematoxylin. To control for the specificity of the primary antibody, increasing amounts of xVICKZ3 recombinant protein was added to the primary antibody before it was used. A decrease in staining intensity was observed as the amount of recombinant protein was increased (data not shown). As a control for the secondary antibody, every sample was stained, in parallel, with BSA instead of the primary antibody and found to give no background staining whatsoever.

A: Lymphoma Cancer Samples

Anti-VICKZ antibody stains lymphomas of GC derived B-cell origin. The VICKZ protein expression pattern in a wide range of lymphoproliferative diseases of the lymph node was analyzed. Table 1, shown hereinbelow, indicates the origin of the different types of lymph and blood tumors.

TABLE 1
Origin of different types of lymphomas.

| Type of lymphoma/leukemia | Description (origin) |
|---|---|
| Precursor B-cell Acute lymphoblastic | Pre-B cell tumor |
| leukemia/lymphoma (Pre-B ALL) | |
| Chronic lymphocytic leukemia (CLL) | Peripheral B-cell tumor in |
| | circulating blood |
| Mantle cell lymphoma | Peripheral B-cell, mantle |
| | zone origin |
| Marginal zone cell lymphoma (also | Peripheral B-cell, marginal |
| called MALT lymphomas) | zone origin |
| Hairy cell leukemia | Peripheral B-cell, unknown |
| | origin |
| Burkitt's lymphoma | Peripheral B-cell, germinal |
| | center (GC) origin |
| Diffuse large B-cell lymphoma | Peripheral B-cell, either GC or |
| (DLBCL) | activated B-cell origins |
| Follicular lymphoma (FL) | Peripheral B-cell, GC origin |
| Plasma cell myeloma | Terminally differentiated B-cells |
| Mediastinal large B-cell lymphoma | Peripheral B-cell, unclear origin |
| | (related to HL?) |
| Hodgkin's lymphoma (HL) | Peripheral B-cell, classic HL |
| | apparent GC origin |
| Precursor T-cell acute lymphoblastic | Pre T-cell tumor |
| leukemia/lymphoma (Pre-T ALL) | |
| Peripheral T-cell neoplasms, including | Peripheral T-cell, |
| anaplastic large cell lymphoma | heterogeneous origins |

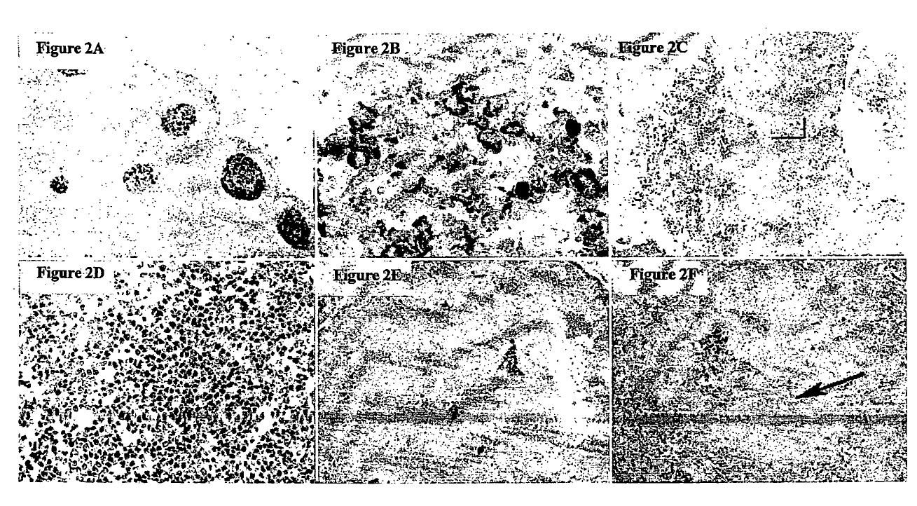

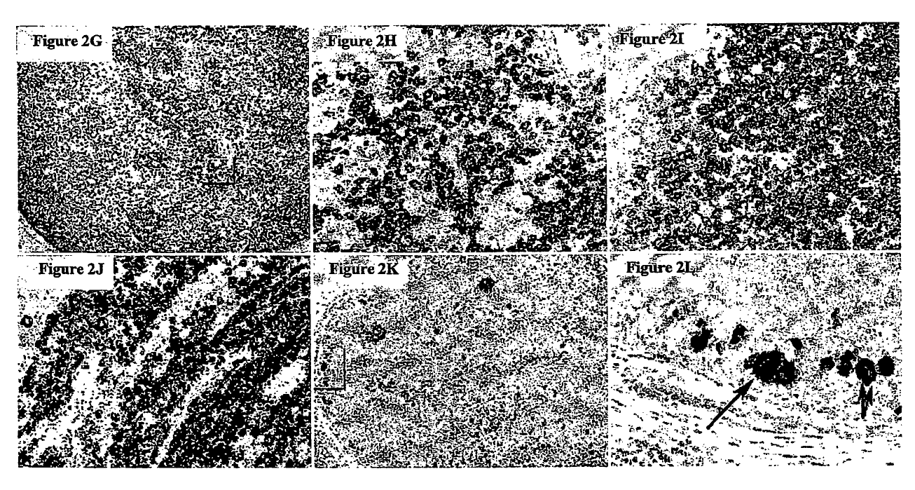

Samples of all of these lymphomas/leukemias were obtained from the archives at the Hadassah Hebrew University Medical Center, Jerusalem. Only those lymphomas with a GC B-cell origin were expected to be VICKZ-positive. This differential expression pattern allows distinction between cutaneous lymphomas of follicular vs. marginal zone origin, as well as classical HL vs. anaplastic large cell lymphoma. Anti-VICKZ staining enables an easier assessment of the grade of follicular lymphomas, which is based on the ratio of centroblasts to centrocytes. Histologically, centroblasts are distinguished from centrocytes primarily on the basis of their larger cytoplasm. The strong, cytoplasmic staining of anti-VICKZ antibody allows easy identification and differentiation of these GC-derived cells; this is especially obvious when compared with cells stained with anti-Bcl-6, the nuclear marker currently used for identifying GC-derived cells (compare anti-VICKZ staining in FIGS. 2A and 2B with anti-Bcl-6 staining in FIGS. 2C and 2D). In cases of fine needle aspirations, when the architecture of the lymph node is not present, the ability to clearly identify GC-derived B-cells greatly improves the ability of pathologists to make a definitive diagnosis, avoiding invasive methods.

FIGS. 2E and 2F, show a histological section of Castleman's disease (an atypical lymphoproliferative disease that has been reported to be associated, in some instances with Kaposi's sarcoma) showing residual GCs, stained red, surrounded by an expanded mantle region, at 4X (E) and 20X (F), with arrow indicating characteristic vascularization. FIGS. 2G, 2H and 2I show immunostaining of tissue whereby VICKZ is highly expressed in follicular lymphomas of all grades (including those of the skin), in classical Hodgkin lymphoma (exclusively in the Reed-Sternberg and Hodgkin cells; FIGS. 2K, 2L), and in DLBCL (FIG. 2J).

Anti-VICKZ Immunostaining is a Prognostic Indicator in DLBCL

Recent microarray analyses have shown that DLBCL develops as two distinct classes, one GC-like and the other ABC-like, based on gene expression signatures (Alizadeh et al., 2000). Three GC markers, including Bcl-6, have been shown to be positively correlated with survival, and three ABC markers, including Bcl-2, have been negatively correlated with survival (Lossos et al., 2004). A retrospective study, analyzing the expression of VICKZ, Bcl-2, and Bcl-6, in approximately 100 cases of DLBCL, compares the expression patterns with the survival rate of the patients. Estimating that there are approximately 30 new cases every year at the Hadassah University hospital, patients for the study are selected from those who in the period 1985-1990 were diagnosed with DLBCL, treated with a regimen of CHOP or CHOP-like compounds, and who were followed up at the hospital. Permission for the study has been requested from the appropriate committee. A univariate Cox proportional-hazards analysis with overall survival as a dependent variable is used to determine hazards rates for each of the genes alone. Those with significant values are used together to generate a model that is tested using the Kaplan-Meier protocol in order to see whether low, medium, and high risk groups can be defined with high predictive power. This approach allows comparison of the prognostic value of VICKZ staining with that of either Bc1-2 or Bcl-6, and also tests whether a combination of these stains provides an even better prognostic indicator.

TABLE 2

| Lymphoma Subtype | Total Positive | % Positive |
|---|---|---|
| B-Cell Lymphoma [N = 439] | | |
| Follicular Lymphoma | 126/165 | 76% |
| Grade 1 | 30/42 | 71% |
| Grade 2 | 45/53 | 85% |
| Grade 3 | 51/70 | 73% |
| Diffuse Large B-cell Lymphoma (DLBCL) | 155/200 | 78% |
| Mediastinal Large B-cell Lymphoma | 9/10 | 90% |
| Burkitt Lymphoma (BL) | 2/2 | 100% |
| Extranodal Marginal Zone Lymphoma | 2/25 | 8% |
| Splenic Marginal Zone Lymphoma | 1/5 | 20% |
| Nodal Marginal Zone Lymphoma | 1/5 | 20% |
| Mantle Cell Lymphoma | 2/18 | 11% |
| Small Lymphocytic Lymphoma/CLL | 3/38 | 8% |
| Lymphoplasmacytic Lymphoma | 0/5 | 0% |
| Precursor B-Lymphoblastic Lymphoma | 4/13 | 25% |
| T-cell Lymphoma [N = 134] | | |
| Precursor T-Lymphoblastic Lymphoma | 4/14 | 29% |
| Peripheral T-cell Lymphoma | 3/21 | 14% |
| Anaplastic Large Cell Lymphoma | 6/8 | 75% |
| NK lymphoma | 2/91 | 2% |
| Plasma Cell Neoplasms [N = 174] | | |
| Multiple Myeloma | 7/153 | 5% |
| Plasma Cell Leukemia | 0/13 | 0% |
| Monoclonal gammopathy (MGUS) | 0/8 | 0% |
| Hodgkin Lymphoma [N = 121] | | |
| Lymphocyte Predominant | 12/13 | 92% |
| Classical Hodgkin | 101/108 | 94% |
| Nodular Sclerosis | 82/85 | 96% |
| Mixed Cellularity | 19/23 | 83% |

Additionally, 868 non-Hodgkin's and Hodgkin's lymphomas were tested by immunohistochemistry on tissue microarrays. Staining for VICKZ protein was present in 76% (126/165) of follicular lymphoma, 78% (155/200) of DLBCL, 90% (9/10) of mediastinal large B-cell lymphoma, and 100% (2/2) of Burkitt's lymphoma. A subset of mantle cell lymphoma (11%, 2/19), extranodal (8%, 2/25), and nodal (20%, 1/5) marginal zone lymphoma and lymphoblastic lymphoma (25%, 4/13), showed VICKZ staining. The majority of lymphocyte predominant Hodgkin (92%, 12/13) and classical Hodgkin (94%, 101/108) lymphoma were found to be positive. Among T cell lymphoma, anaplastic large cell lymphoma were positive (75%, 6/9). Table 2 hereinabove presents results of immunostaining of VICKZ in lymphoma subtypes. Cases were scored positive if more than 30% of the lymphoma cells stained for VICKZ.

The differential expression pattern of VICKZ protein in lymphoma subtypes confirms utility in identifying VICKZ expression for the differential diagnosis of DLBCL associated with different prognoses.

B: Colorectal Cancer Samples

For the Hadassah cohorts, paraffin blocks of colon tumors were collected from the archives of the Department of Pathology at the Hadassah Hebrew University Medical Center. Experiments using human tissue received IRB exemption by the IRB chair. Resected CRC tissue samples from patients (from the years 1999-2004) who had not undergone neoadjuvant therapy were chosen randomly from the archive. Antibody-stained samples were scored by three independent observers. A score from 0 to 3 was given to each sample according to the average intensity of all of the tumorous cells, with 0 indicating no detectable staining, 1, barely detectable staining, 2, a clear staining, and 3, the strongest observed stain. The scores of the three observers were averaged for every sample. The observers were blinded, during the evaluation process, with respect to any clinical information about the sample.

Results: VICKZ Expression During the Progression of CRC Tumors and Metastases

To further explore the role of VICKZ proteins in cancer, we focused on the pattern of VICKZ expression as a function of CRC progression. CRC ranks among the top four cancer killers, with approximately 148,300 new diagnoses and over 55,000 deaths a year in the US alone. A sequence for the development of the large majority of these cancers has been described in detail, beginning with benign polyps (adenomas) that jut into the lumen of the gut, acquire additional mutations, and eventually grow inwards to develop into adenocarcinomas (Markowitz, et al, 2002). These neoplastic cells can invade the underlying submucosa, muscularis, and serosa layers and form clonal metastases in the adjacent lymph nodes. Ultimately, the growths become dispersed, via the lymphatics and blood vessels, to distal sites. As summarized hereinbelow in Table 1, we have analyzed expression in 139 resected, archived samples from 62 patients treated from 2000-2004 at Hadassah Hospital in Jerusalem, Israel; in 31 of these patients, metastases in lymph nodes have been tested. An example of an adenocarcinoma that developed on the background of an adenoma is shown in FIG. 2A, which contains a section of a polyp protruding into the lumen of the colon, along with the underlying muscularis and serosa layers. This sample shows atypical progression for a CRC tumor with the different stages, all from the same patient, highlighted in FIGS. 2A and 2F and shown in higher magnification in FIGS. 2B′-E″ and 2G′-G″. Generally, in normal colonic epithelia and surrounding tissues, VICKZ expression is essentially non-detectable (FIGS. 2B, B′, B″). In tubulovillous adenomas, VICKZ expression becomes detectable, particularly in areas of more notable dysplasia, although the expression level is low (FIGS. 2C, C, C″). As the neoplastic cells invade the tissue, a striking, graded pattern of expression is often observed (FIG. 2D, D′, D″), with the invasive edge of the neoplasia, that is advancing through the subserosa, demonstrating the strongest staining (FIGS. 2E, E′, E″). Lymph node metastases are strongly positive, and the surrounding normal T cells completely negative, for VICKZ expression (FIGS. 2F, G, G′, G″). Thus, CRC progression is characterized by elevated levels of VICKZ protein.

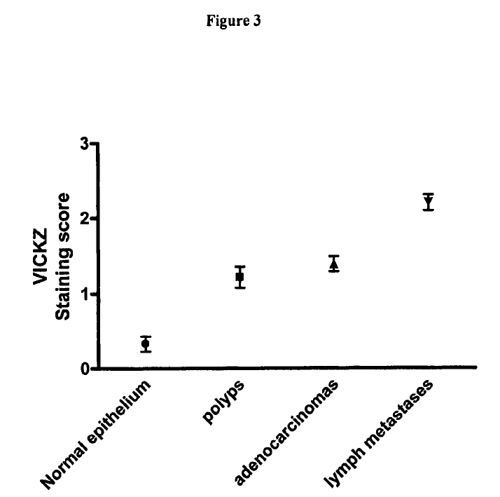

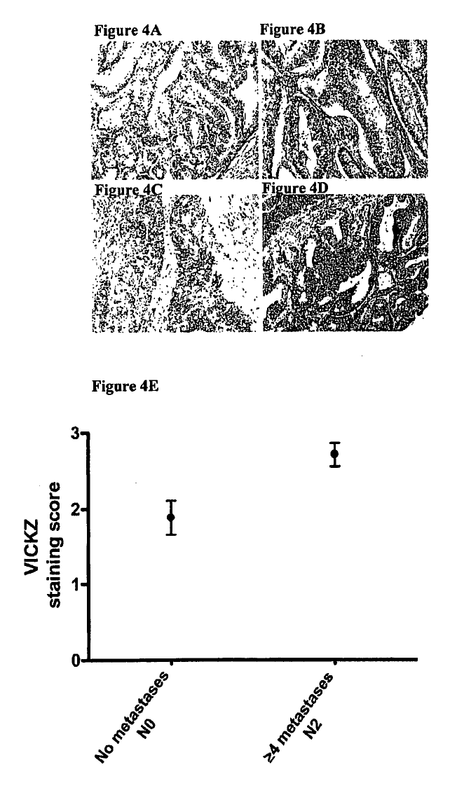

The samples were scored based on the average relative intensity of VICKZ expression in the neoplastic cells. Approximately 67% (56 out of 84) of the adenomas or adenocarcinomas from the Hadassah Medical Center study were positive for VICKZ expression. Despite the fact that one third of the primary growths or tumors, in both studies, scored negative for VICKZ expression, we find that a striking 90% (54 out of 60) of the lymph node metastases show moderate to high VICKZ levels, using the same scoring system described above. The level of expression of the proteins is also significantly higher (P<0.001) in the lymph node metastases than in the normal surrounding colonic epithelium, adenomas, or adenocarcinomas, as judged by one way ANOVA analysis using a Kruskal-Wallis test (FIG. 3). Given the gradient of VICKZ expression i n invasive adenocarcinomas, and the widespread and high levels of VICKZ expression in lymph node metastases, these results suggest that VICKZ proteins may play a role in helping to mediate metastasis in CRC. To test this hypothesis, we analyzed, in a double-blind experiment, an additional 25 samples from patients who had invasive CRC (stage T3) to see whether their VICKZ levels correlated with metastasis (Table 3B). Patients with n o mesenteric lymph node metastases (NO) had significantly (P=O.037) lower VICKZ expression in their primary tumors than those with four or more metastases (NT), which is a known marker of poor outcome (FIGS. 4C and 4D). Thus, VICKZ expression levels in the primary tumor are a predictor of the extent of lymph node metastases, and therefore, a potentially valuable prognostic indicator.

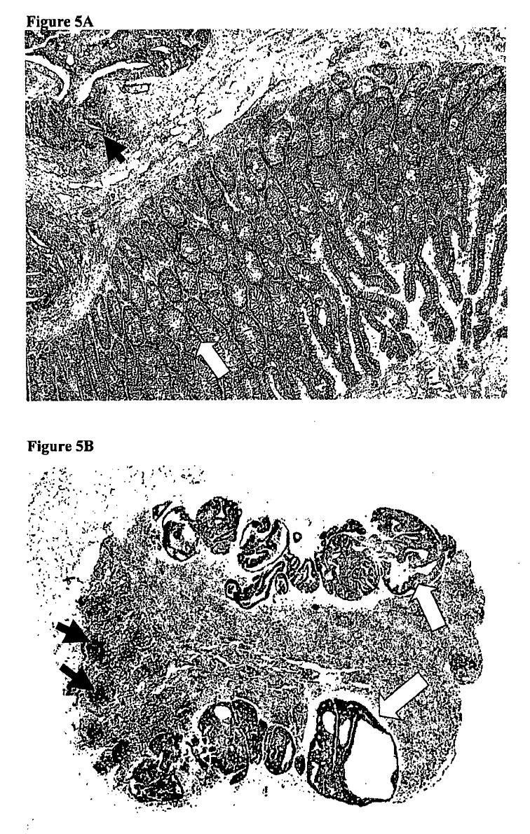

FIG. 5A shows staining of moderately differentiated adenocarcinoma of the colon. The antibody specifically stains the adenocarcinomas (dark arrow) while the normal epithelia and surrounding tissue are completely negative (white arrow). FIG. 5B shows staining of a lymph node metastasis from a moderately differentiated adenocarcinoma. Although the metastases stain strongly for VICKZ protein expression, the adjacent normal lymphoid tissue does not express the proteins, with the notable exception of the germinal centers (black arrows).

### Example 5

### VICKZ Expression in Transfected Cells

The intracellular localization of VICKZ proteins during cell migration was shown using the SW480 colorectal carcinoma cell line.

Materials and Methods

The ORF of each of the VICKZ isoforms was cloned into the expression vector pEGFP-Cl (Clontech), to generate pEGFP-hVICKZ1, pEGFP-hVICKZ2, pEGFP-hVICKZ3 and pEGFP-xVICKZ3.

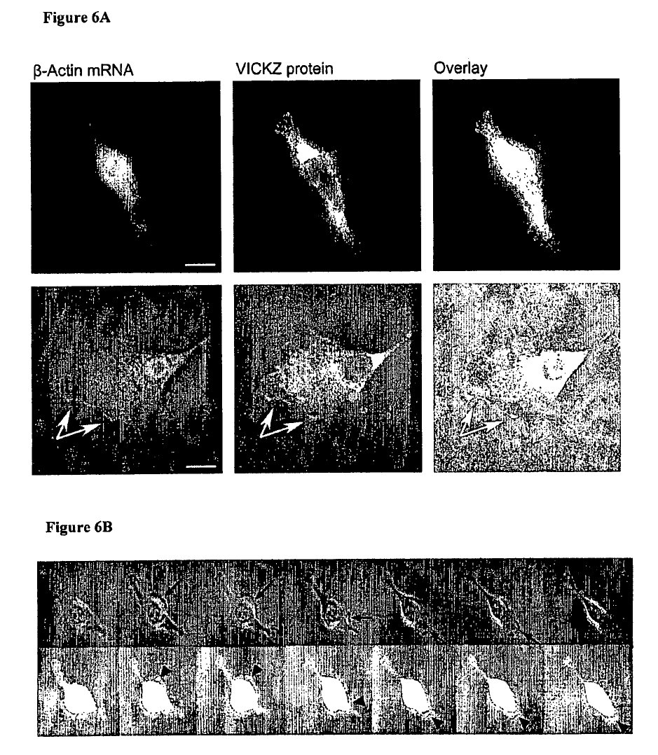

Sub-confluent SW480 cells were serum starved for 6 hours. To induce cells, PMA (Sigma) was added to a final concentration of 100 ng/ml. After one hour, uninduced and induced cells were fixed and standard in situ hybridization was performed. Cy-3 conjugated human β-actin probe was a kind gift of Dr. Robert Singer. Following in situ hybridization, cells were washed three times with 1×PBS, 5 mM MgCl₂ and blocked with CAS-block supplemented with 1 mg/ml RNAse free BSA (NEB) for an hour. Affinity-purified xVICKZ3 antibody (1:100) was added to blocking solution and incubated O.N. at 4° C. The next day the cover slips were washed with 1×PBS, 5 mM MgCl₂, incubated with 1:100 anti-rabbit Cy-5 conjugated antibody (Jackson) for 1 hour at R.T., washed again three times, and mounted. Raw black & white images were processed by 2-D convolution using the AutoDeBlur software (AutoQuant, Inc.) and pseudo-colored with Adobe Photoshop. FIG. 6A upper row shows starved SW480 cells were induced by PMA and fixed after 60 minutes (FIG. 6A, lower row). Fluorescence in-situ hybridization with a mRNA probe (rhodamine-labeled) was then performed, followed by indirect immunofluorescence using the pan-VICKZ antibody as the primary antibody and an anti-rabbit, Cy-5-conjugated, secondary antibody. Deconvolution was performed using 2-D algorithm (AutoDeblur software, AutoQuant, Inc.). Intracellular localization of β-actin mRNA is shown in gray and intracellular localization of VICKZ protein is shown in light gray. Localization of β-actin transcripts and VICKZ protein to the lamellar leading edge (lamellipodia) is observed in induced cells and is indicated by white arrows. Overlay image of β-actin mRNA and VICKZ protein shows colocalization in white.

Time Lapse Microscopy

SW480 cells transiently transfected with pEGFP-hVICKZ1 were seeded on laminin-coated cover slips. Cells were induced as described above. Images were collected at 5 minutes intervals at 37° C. by a cooled CCD camera (Sensicam; PCO imaging) that was mounted on an inverted Axiovert 200 microscope (Carl Zeiss Microimaging, Inc.) equipped with motorized stage and X63 air lens (Zeiss). Images were acquired using Image Pro (Media Cybernetics, Inc.). SW480 cells were transfected with GFP-hVICKZ1 plasmid and seeded on lamin-coated cover slips. After two days, the cells were induced as described. Fluorescent time-lapse microscopy was used to study the cellular distribution of GFP-hVICKZ1 over time within living cells. Pictures were taken every 5 minutes. Upper row, phase contrast series of the cell shows the lamella as a dark structure at the edge of the cell. Black arrows point to the area where the lamella is most active. Lower row, corresponding fluorescent images show that GFP-hVICKZ1 clearly localizes to the protruding, well-developed lamella. The last three pictures show that as the lamella disappears, the GFP-hVICKZ1 is delocalized from the cell edge, illustrating spatial as well as temporal localization. Because the fluorescent images were overexposed in order to detect the signal in the lamella, the nucleus in these pictures is masked by the overlying cytoplasmic signal.

VICKZ proteins localize, upon induction of cell movement, to lamellipodia in the colorectal carcinoma-derived SW480 cells (FIGS. 6B). β-actin mRNA co-localizes with the proteins in these migrating cells (FIG. 6A). Although co-localization of VICKZ proteins and β-actin mRNA has been previously reported in normal, migrating chick embryo fibroblasts and in dendrites the results presented here represent, to the best of our knowledge, the first report of such localization in motile, neoplastic cells. The correlation between VICKZ expression, lamellipodia localization, and cell movement may be a very tumor-specific phenomenon. In studies comparing invasive and non-invasive rat breast cancer cell lines, motility and metastasis were found to correlate with a lack of β-actin mRNA localization (Shestakova et al, 1999) and a down-regulation of rVICKZ1 (Wang et al. 2002) In fact, it was recently postulated that VICKZ1 may act as an anti-metastatic factor (Condeelis and Singer, 2005). The results herein indicate that, at least in the case of colorectal carcinomas, VICKZ proteins are actually pro-metastatic factors.

### Example 6

### Inhibition of VICKZ with Expressed siRNAs VICKZ Specific RNAi Molecules

The different RNAi molecules known in the art include antisense RNA and antisense DNA, double stranded RNAi, siRNA, micro-RNA and ribozymes. Specific nucleic acids are targeted for RNA interference. “Targeting” typically begins with identification of a nucleic acid sequence whose function i s to be modulated. In the present invention, the target is VICKZ mRNA. The targeting process also includes determination of a site or sites within this mRNA for the RNAi interaction to occur such that the desired effect, e.g., interference of translation, will result in specific suppression of VICKZ expression.

RNAi sequences are identified using algorithms known to those with skill in the art including, in a non-limiting example, OptiRNAi computer program. US patent application 20040072769 teaches methods for design and selection of short double-stranded oligonucleotides.

Expression of siRNAs from viral and non-viral vectors offers several advantages over synthetic siRNAs, such as stable selection under selectable markers and inducible promoters, which are features that could be useful for genetic approaches to therapy. Expressed siRNAs are tested for their ability to inhibit VICICZ.

Plasmids are constructed containing a 19-30 base pair (bp) region of the VICKZ gene in 5′-3′ and 3′-5′ orientations under the control of a T7 promoter. VICKZ expression was followed in cancer cells transfected with VICKZ-YFP vector cells and a vector expressing T7 RNA polymerase (T7 pol). In the presence of T7 RNA polymerase, T7 transcripts derived from linearized expression plasmids comprise the 19-30 bp VICKZ sequence and are expected to inhibit VICKZ expression. By comparison, the presence of an identical plasmid lacking VICKZ sequences has no effect on VICKZ production in co-transfected cells.

Lentivirus vectors have been tested as expression vectors for siRNA.

siRNA constructs are prepared and cloned into lentiviral vectors that result in the reduction of expression of any or all of the VICKZ isoforms. Alternatively, constructs comprising dominant negative forms of VICKZL proteins that have been proven effective at inhibiting VICKZ activity in vitro and in mice in vivo are prepared. The constructs are under tet regulation in order t o tightly control the levels of the siRNA or dominant negative protein. (Taulli et al, 2005; Vigna, et al, 2005).

### Example 7

### Preparation of a Dominant Negative Construct

In a parallel approach, taking advantage of the fact that VICKZ proteins bind RNA as a dimer (Git and Standart, 2002), a deletion in the last KH domain of VgI RBP that dominantly inhibits VgI RBP binding to RNA targets was generated.

The dominant negative constructs were prepared by cloning the xenopus or human VICKZ DNA lacking the nucleotides encoding the KH4 domain into suitable vectors. The DNA encoding the open reading from of human VICKZ1 lacking the KH4 domain is referred to herein “hΔKH4α” and is set forth in SEQ ID No: 14. The corresponding human amino acid sequence is set forth in SEQ ID NO: 15. Tie vector, which includes the vector sequences and the DNA encoding the dominant negative xenopus protein is referred to herein “xΔKH4α” and is set forth in SEQ ID NO: 16.

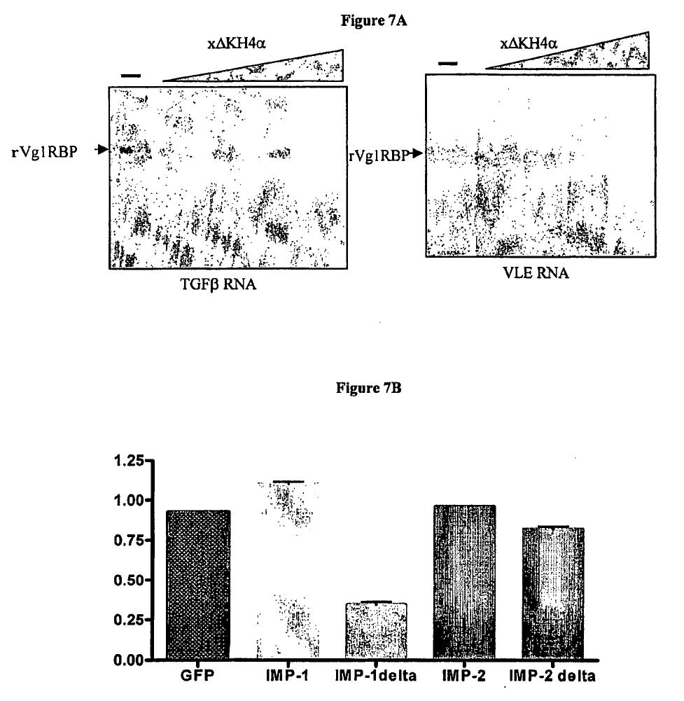

In Xenopus embryos, injection of this construct phenocopies the inventors' previously reported effects of antisense morpholino oligonucleotides directed against VgI RBP, specifically inhibition of cell migration of the dorsal fin, head, lens and lateral pigment cells. FIG. 7A: recombinant VgI RBP (rVg1 RBP) or oocyte extract containing endogenous VgI RBP was UV-crosslinked to either a radioactively labeled VgI RNA localization element probe (VLE) or to a TGFβ probe in the presence of increasing amounts of recombinant xΔKH4α and then processed and electrophoresed on an SDS-polyacrylamide gel. Essentially equimolar and higher concentrations of xΔKH4α severely reduce VgI RBP binding activity.

The corresponding deletion in the human IMP isoforms was generated and tested for their effects on cell migration. As seen in FIG. 7B, transfection of the dominant negative hΔKH4α construct into PC3 (metastatic prostate carcinoma cell line) cells induced to migrate by EGF causes a significant drop in cell motility, compared with either transfected full length IMP-I or GFP. These results indicate that cell migration, in these carcinoma cells, requires the activity of full length VICKZ proteins.

### Example 8

### Preparation of an Anti-VICKZ Vaccine

In brief, peripheral blood Dendritic Cells (DC) precursors are purified from peripheral blood mononuclear cells obtained at leukapheresis by density gradient centrifugation steps and then cultured for 2 days with VICKZ protein (either coupled or not coupled to KLH to enhance immunogenicity) to allow the cells to take up the antigen as they undergo maturation and activation. The mature, antigen-loaded DCs are then washed and administered intravenously.

### REFERENCES

Alizadeh, A A, Eisen, M B, Davis, R E, Ma, C , Lossos, I. S., Rosenwald, A., Boldrick, J. C , Sabet, H., Tran, T., Yu, X, et al (2000). Distinct types of diffuse large B-cell lymphoma identified by gene expression profiling. Nature 403, 503-511.

Ausubel, et al (Eds), Current Protocols in Molecular Biology, John Wiley & Sons, Inc. (New York) (1987-1999)

Condeelis J, Singer R H (2005) How and why does beta-actin mRNA target? Biol Cell. 97(1):97-110.

Colligan, et al (eds.), Current Protocols in Immunology, John Wiley & Sons, Inc. (New York) (1992-2000)

Doyle, G. A., Betz, N. A., Leeds, P. F., Fleisig, A. J., Prokipcak, R. D., and Ross, J. (1998). The c-myc coding region determinant-binding protein: a member of a family of KH domain RNA-binding proteins. Nucleic Acids Res 26, 5036-5044.

Doyle, G. A., Bourdeau-Heller, J. M., Coulthard, S., Meisner, L. F., and Ross, J. (2000). Amplification in human breast cancer of a gene encoding a c-myc mRNA-binding protein. Cancer Res 60, 2756-2759.

Gennaro (ed.), Remington's Pharmaceutical Sciences, 18^{th} Ed. (Mack Publishing Co., 1990)

Harlow, et al, Antibodies: A Laboratory Manual, CSHL (Cold Spring Harbor, NY) (1988)

Havin, L., Git, A., Elisha, Z., Oberman, F., Yaniv, K., Schwartz, S. P., Standart, N., and Yisraeli, J. K. (1998). RNA-binding protein conserved in both microtubule- and microfilament-based RNA localization. Genes Dev 12, 1593-1598.

Kohler, G. and Milstein, C, (1975) Continuous cultures of fused cells secreting antibody of predefined specificity. Nature, 256(55 17):495-497.

Kuppers, R. (2002). Molecular biology of Hodgkin's lymphoma. Adv Cancer Res 84, 277-312.

Li L, Thomas RM₃ Suzuki H, De Brabander J K, Wang X, Harran P G. A small molecule Smac mimic potentiates TRAIL- and TNF alpha-mediated cell death. 2004. Science.305(5689): 1471-4.

Liao, B. Liao B, Patel M, Hu Y, Charles S, Herrick D J, Brewer G. 2004. Targeted knockdown of the RNA-binding protein CRD-BP promotes cell proliferation via an IGF-II-dependent pathway in human K562 leukemia cells. J Biol Chem. Sep 8 [Epub ahead of print]

Liu A Y, Robinson R R, Hellstrom K E, Murray E D Jr, Chang C P, Hellstrom I. (1987). Chimeric mouse-human IgG1 antibody that can mediate lysis of cancer cells. PNAS USA, 84(10):3439-3443

Lu, M., Nakamura, R. M., Dent, E. D., Zhang, J. Y., Nielsen, F. C₅ Christiansen, J., Chan, E. K., and Tan, E. M. (2001). Aberrant expression of fetal RNA-binding protein p62 in liver cancer and liver cirrhosis. Am J Pathol 159, 945-953.

Markowitz S D, Dawson D M, Willis J, Willson JK . (2002) Focus on colon cancer. Cancer Cell 1(3):233-6.

Muller K M, Arndt K M, Pluckthun A. (1998). A dimeric bispecific miniantibody combines two specificities with avidity. FEBS Letters, 432:45-49.

Mueller F, Bommer M, Lacher U, Ruhland C, Stagge V, Adler G, Gress T M, Seufferlein T. (2003) KOC is a novel molecular indicator of malignancy. Br J Cancer. 58(5), 699-701.

Rosenwald, A., Wright, G., Chan, W. C 5 Connors, J. M., Campo, E., Fisher, R. L, Gascoyne, R. D., Muller-Hermelink, H. K., Smeland, E. B., Giltnane, J. M, et al. (2002). The use of molecular profiling to predict survival after chemotherapy for diffuse large-B-cell lymphoma. N Engl J Med 346, 1937-1947.

Ross, J., Lemm, L₅ and Berberet, B. (2001). Overexpression of an niRNA-binding protein in human colorectal cancer. Oncogene 20, 6544-6550.

Sambrook, J, Fritsch, E. F. and Maniatis, T. (1989) Molecular Cloning: A laboratory manual, Cold Spring Harbor Laboratory Press, Cold Spring Harbor, USA.

Stewart, S A, Dykxhoom, D M, Palliser, D, Mizuno, H₅Yu₅EY, An, DS₅ Sabatini, D M, Chen, I S Y, Hahn, W C, Sharp, P A₅ Weinberg, R A and Novina, C D. (2003) Lentivirus-delivered stable gene silencing by RNAi in primary cells. RNA 9:493-501.

Taulli R₅Accornero P₅Follenzi A₅Mangano T₅Morotti A₅Scuoppo C,Forni P E, Bersani F, Crepaldi T₅Chiarle R₅Naldini L₅Ponzetto C. (2005) RNAi technology and lentiviral delivery as a powerful tool to suppress Tpr-Met-mediated tumorigenesis. Cancer Gene Ther. May;12(5):456-63.

Ui-Tei, K., Naito, Y., Takahashi, F., Haraguchi, T., Ohni-Hamazaki, H., Juni, A., Ueda,, R., and Saigo, K. (2004). Guidelines for the selection of highly effective siRNA sequences for mammalian and chick RNA interference. NAR 32(3ˆ:936-48.

Vigna E, Amendola M, Benedicenti F, Simmons A D, Follenzi A, Naldini L. (2005) Efficient Tet-dependent expression of human factor IX in vivo by a new self-regulating lentiviral vector. Mol Ther. May;11(5):763-75.

Walensky L D, Kung A L, Escher I, Malia T J, Barbuto S, Wright R D, Wagner G, Verdine G L, Korsmeyer S J. Activation of apoptosis in vivo by a hydrocarbon-stapled BH3 helix. 2004. Science. 305(5689): 1466-70.

Wang W, Wyckoff J B, Frohlich V C, et al (2002). Single cell behavior in metastatic primary mammary tumors correlated with gene expression patterns revealed by molecular profiling. Cancer Res 62(21):6278-88.

Yaniv, K., Fainsod, A., Kalcheim, C, and Yisraeli, J. K. (2003). The RNA-binding protein VgI RBP is required for cell migration during early neural development. Development 130, 5649-5661.

Yaniv, K., and Yisraeli, J. K. (2002). The involvement of a conserved family of RNA binding proteins in embryonic development and carcinogenesis. Gene 287, 49-54.

Yantiss R K, Woda B A, Fanger G R, Kalos M, Whalen G F, Tada H, Andersen D K, Rock K L, Dresser K. (2005) KOC: a novel molecular marker that distinguishes between benign and malignant lesions of the pancreas. Am J Surg Pathol. 29(2): 188-95.

Zhang, J. Y., Chan, E. K., Peng, X. X., Lu, M., Wang, X., Mueller, F., and Tan, E. M. (2001). Autoimmune responses to mRNA binding proteins p62 and Koc in diverse malignancies. Clin Immunol 100, 149-156.

Zhou A, Stein P E, Huntington J A, Sivasothy P, Lomas D A, Carrell. 2004. RW. How small peptides block and reverse serpin polymerisation. J Mol Biol. 342(3) :93 1-41.

Citation of any document herein is not intended as an admission that such document is pertinent prior art, or considered material to the patentability of any claim of the present application.

What is claimed is:

1. A method for the diagnosis of a disorder selected from the group consisting of lymphoma and metastatic disease in a subject, the method comprising the step of detecting the presence of a VICKZ expression product in a suitable biological specimen from the subject.

2. The method of claim 1, said method comprising the steps of
a. contacting a suitable biological specimen from the subject with a molecule having specific affinity to a VICKZ expression product; and
b. detecting whether the molecule binds to the specimen;
wherein detection of binding between said specimen and said molecule indicates a positive diagnosis of a disorder selected from the group consisting of lymphoma and metastatic disease.

3. The method according to claim 2 wherein the expression product is a VICKZ protein and the molecule is an antibody having specific activity for at least one member of the family of VICKZ proteins.

4. The method according to claim 2 wherein the expression product is VICKZ RNA and the molecule is a nucleic acid molecule having sequence complementary to at least a part of at least one member of the family of VICKZ polynucleotides.

5. The method according to claim 1 wherein the lymphoma is germinal center B-cell derived lymphoma.

6. The method of claim 3 wherein the antibody is selected from the group consisting of a polyclonal antibody, a monoclonal antibody, a proteolytic fragment of an antibody, a chimeric antibody and a recombinant antibody.

7. The method of claim 6 wherein the antibody is a polyclonal antibody.

8. The method of claim 7 wherein the polyclonal antibody is anti-pan-VICKZ raised to the Xenopus VG1 RBP polypeptide variant D having an amino acid sequence as set forth in SEQ ID NO:1.

9. The method of claim 8 wherein the anti-pan-VICKZ antibody is an affinity purified antibody.

10. The method according to claim 3 wherein the antibody is raised to a human peptide selected from the group consisting of a human VICKZ1 peptide having SEQ ID NO:2; a human VICKZ2 peptide having SEQ ID NO:3 and a human VICKZ3 peptide having SEQ ID NO:4.

11. The method according to claim 4, wherein the VICKZ nucleic acid molecule is selected from the group consisting of VICKZ antisense RNA or a fragment thereof, VICKZ cDNA or a fragment thereof and a VICKZ-specific oligonucleotide primer.

12. The method of claim 1 wherein the metastatic disease is selected from the group consisting of colorectal cancer, prostate cancer, ovarian cancer, non-small cell lung cancer and hepatocellular cancer

13. The method according to claim 1 wherein the suitable biological specimen from a subject is selected from the group consisting of colorectal tissue, lymph node, plasma and bone marrow.

14. The method according to claim 13 wherein the suitable biological sample is lymph node tissue.

15. A method for preventing or treating a disorder selected from the group consisting of lymphoma and metastatic disease, the method comprising administering to a subject in need thereof a composition comprising a therapeutically effective amount of at least one VICKZ specific inhibitor and a pharmaceutically acceptable carrier.

16. The method of claim 15 wherein the VICKZ inhibitor is selected from the group consisting of an RNA interference (RNAi) molecule, a peptide analog and a dominant negative protein.

17. The method of claim 16 wherein the VICKZ inhibitor is an RNA interference (RNAi) molecule.

18. The method of claim 17 wherein the RNAi is selected from the group consisting of dsRNA, siRNA, antisense RNA, micro RNA and a ribozyme.

19. The method of claim 15 wherein the specific VICKZ inhibitor is a peptide analog having an amino acid sequence derived from the VICKZ polypeptide sequence.

20. The method of claim 19 wherein the peptide analog is selected from the group consisting of a cyclic peptide, a linear peptide and a peptidomimetic.

21. The method of claim 15 wherein the lymphoma is Germinal Center B cell derived lymphoma

22. A method for preventing or treating a disorder selected from the group consisting of lymphoma and metastatic disease, the method comprising administering to a subject in need thereof an immunologically effective amount of at least one molecule selected from the group consisting of a VICKZ protein, an immunogenic fragment of a VICKZ protein, a nucleic acid that encodes a VICKZ protein and a nucleic acid that encodes a fragment of a VICKZ protein;
whereby an immune response is generated to said protein.

23. A pharmaceutical composition comprising as an active ingredient at least one VICKZ specific inhibitor, and a pharmaceutically acceptable diluent or excipient.

24. The pharmaceutical composition of claim 23 wherein the specific VICKZ inhibitor is selected from the group consisting of an RNA interference (RNAi) molecule, a peptide analog and a dominant negative protein.

25. The pharmaceutical composition of claim 24 wherein the RNAi molecule is selected from the group consisting of dsRNA, siRNA, antisense RNA, micro RNA and a ribozyme.

26. The pharmaceutical composition of claim 23 wherein the specific VICKZ inhibitor is a peptide analog having an amino acid sequence derived from the VICKZ polypeptide sequence.

27. The pharmaceutical composition of claim 26 wherein the peptide analog is selected from the group consisting of a cyclic peptide, a linear peptide and a peptidomimetic.

28. A pharmaceutical composition comprising: (a) an antigen-presenting cell that expresses a VICKZ polypeptide or peptide and (b) a pharmaceutically acceptable carrier or excipient.

29. The pharmaceutical composition of claim 28 further comprising an immunostimulant.

30. The pharmaceutical composition of claim 28 wherein the antigen-presenting cell is selected from the group consisting of dendritic cells, macrophages, monocytes, fibroblasts and B cells.

31. A kit for the diagnosis of germinal center B cell lymphoma, the kit comprising
a. a binding molecule specific for a VICKZ expression product; and
b. a means for detecting whether the specific binding molecule is bound to the VICKZ expression product.

32. A kit for the diagnosis of metastatic disease, the kit comprising:
a. a binding molecule specific for a VICKZ expression product; and
b. a means for detecting whether the specific binding molecule is bound to the VICKZ expression product.

33. The kit according to claim 32 wherein the metastatic disease is selected from the group consisting of colorectal cancer, prostate cancer, ovarian cancer, non-small cell lung cancer and hepatocellular cancer.

34. The kit according to claim 33 wherein metastatic disease is colorectal cancer.


SEQUENCE LISTING (16 sequences)

SEQ ID NO: 1 - protein, 594 aa, Xenopus laevis
  ref <301>: AUTHORS: Havin,L. Git,A. Elisha,Z. Oberman,F. Yaniv,K. Pressman Schwartz,S. Standart,N.M. Yisraeli,J.K.
  ref <302>: TITLE: RNA binding protein conserved in both microtubule and microfilament-based RNA localization
  ref <303>: JOURNAL: Genes and Development
  ref <304>: VOLUME: 12
  ref <305>: ISSUE: 11
  ref <306>: PAGES: 1593-1598
  ref <307>: DATE: 1998-05-11
  ref <308>: DATABASE ACCESSION NUMBER: AAC18598
  ref <309>: DATABASE ENTRY DATE: 1998-06-03
  ref <313>: RELEVANT RESIDUES: (1)..(594)
MNKLYIGNLSENVSPTDLESLFKESKIPFTGQFLVKSGYAFVDCPDETWAMKAIDTLSGKVELHGKVIEVEHSVPKRQRSRKLQIRNIPPHLQWEVLDSLLAQYGTVENCEQVNTESETAVVNVTYANKEHARQGLEKLNGYQLENYSLKVTYIPDEMATPQAPSQQLQQQPQQQHPQGRRGFGQRGPARQGSPGAAARPKPQTEVPLRMLVPTQFVGAIIGKEGATIRNITKQTQSKIDIHRKENAGAAEKPITIHSTPEGCSAACKIIMEIMQKEAQDTKFTEEIPLKILAHNNFVGRLIGKEGRNLKKIEQDTDTKITISPLQDLTLYNPERTITVKGSIEPCAKAEEEIMKKIRESYENDIAAMNLQAHLIPGLNLNALGLFPSSSSGMPPPSVGVPSPTSSTSYPPFGQQPESETVHLFIPALAVGAIIGKQGQHIKQLSRFAGASIKIAPAEGPDAKLRMVIITGPPEAQFKAQGRIYGKLKEENFFGPKEEVKLETHIKVPSYAAGRVIGKGGKTVNELQNLTSAEVVVPRDQTPDENDEVVVKITGHFYASQLAQRKIQEILAQVRRQQQQQQKTVQSGQPQPRRK

SEQ ID NO: 2 - protein, 13 aa, Artificial
  synthetic peptide
GCHQKGQSNQAQA

SEQ ID NO: 3 - protein, 16 aa, Artificial
  synthetic peptide
GCEQKYPQGVASQRSK

SEQ ID NO: 4 - protein, 13 aa, Artificial
  synthetic peptide
GCQKALQSGPPQS

SEQ ID NO: 5 - RNA, 2405 nt, Xenopus laevis
  ref <301>: AUTHORS: Havin,L. Git,A. Elisha,Z. Oberman,F. Yaniv,K. Pressman Schwartz,S. Standart,N.M. Yisraeli,J.K.
  ref <302>: TITLE: RNA binding protein conserved in both microtubule and microfilament-based RNA localization
  ref <303>: JOURNAL: Genes and Development
  ref <304>: VOLUME: 12
  ref <305>: ISSUE: 11
  ref <306>: PAGES: 1593-1598
  ref <307>: DATE: 1998-05-11
  ref <308>: DATABASE ACCESSION NUMBER: AF064634
  ref <309>: DATABASE ENTRY DATE: 1998-06-03
  ref <313>: RELEVANT RESIDUES: (1)..(1785)
  ref <301>: AUTHORS: Havin,L. Git,A. Elisha,Z. Oberman,F. Yaniv,K. Pressman Schwartz,S. Standart,N.M. Yisraeli,J.K.
  ref <302>: TITLE: RNA binding protein conserved in both microtubule and microfilament-based RNA localization
  ref <303>: JOURNAL: Genes and Development
  ref <304>: VOLUME: 12
  ref <305>: ISSUE: 11
  ref <306>: PAGES: 1593-1598
  ref <307>: DATE: 1998-05-11
  ref <308>: DATABASE ACCESSION NUMBER: AF064634
  ref <309>: DATABASE ENTRY DATE: 1998-06-03
  ref <313>: RELEVANT RESIDUES: (1)..(2405)
gaauuccccgggggugccuacauuaacguagagugacauuuucacagucugccugccaaaaggauuugggggaagacaaaaaguuguuuuuuuuuuccuccuccuuuuuuuuuuuucuuucuaagcccgccauucuaguaagagggaaaaaggaaucuguuuugccccuuuuccuccugcacauccggacaacacgcgaaauugcucuucuuucccucucagcucucgcuuuugagucgagcagcgacuacagaccugauuuacugguuccucucgaucgccgcccccugauucacugcucaagcugcucggguuuuucgggcucgucuucuuuuagcuuuuuauuauuauuuuuuuuauuuucauuuuuuuuuuacaaugaacaagcuguauauuggaaaccucagcgaaaacgugagccccacggauuuggaaagucucuucaaggagucuaagaucccuuucaccgggcaguuucuggucaaaaguggauacgcguuuguggacuguccggaugagacgugggcuaugaaggccaucgacacccucucagggaaaguggaguugcauggcaaagugauugaagucgaacacucgguacccaaaaggcaaaggagucgaaagcuucagauacgaaacauaccgccucaucuacagugggagguacuggacagccuguuggcacaguaugggacaguggaaaacugugagcaagugaacacugaaucagaaacugcaguagugaauguaacauaugccaauaaggagcacgcuagacaaggacucgaaaaauuaaacggcuaucagcuggaaaacuauagccugaaagugacguauauaccugaugaaauggcaacaccgcaagcaccaucccaacagcuucagcagcagccgcagcagcaacacccucaagggaggcguggguuugggcaaaggggaccugccaggcagggauccccuggugcugcugcaagaccuaagccucagacagagguuccgcugagaaugcugguucccacacaguuuguaggggcgaucauugggaaagagggugcuaccaucaggaacaucacuaaacagacccagucaaaaauagauauucaccguaaagaaaaugcaggugcugcagagaaaccgauuacaauucacucaaccccugaaggcugcucagcugcauguaagaucauuauggagaucaugcagaaggaagcucaggauaccaaguucacugaagaaauccccuuaaaaauauuagcacauaacaacuuuguuggccgucuuauugguaaagaaggaaggaaccuuaagaaaauugagcaagauacagauacaaaaaucacaauaucuccacuacaggacuugacacuguacaauccagaacggaccauuacaguaaaaggcagcauugagccaugugccaaagccgaggaagaaaucaugaaaaaaauuagggaguccuaugaaaaugauauugcugcuaugaaucuccaggcacauuugauuccugguuugaaccugaaugcauugggccuuuucccaucauccucaucaggaaugccaccaccuucuguuggaguucccucaccuaccucaucuacuucuuauccaccauuugggcagcagccagagucagagaccguucaucucuucaucccagcuuuagcugugggagcgauuauuggaaagcaaggacaacacaucaaacaacuaucacguuuugcaggagcaucuauuaagauugcaccugcggagggaccugaugccaaacuaagaauggugaucaucacuggaccuccugaagcccaauuuaaggcucaaggaaggaucuaugguaaacugaaagaagaaaacuucuucggaccuaaagaagaagugaaacuugagacccacauuaaagugccgucauaugcugcuggacggguuauuggcaaaggaggaaaaacaguaaaugaacuucagaauuuaacaagugcagagguuguugugccccgugaucaaacaccagaugaaaaugaugaaguggucguuaagauaacgggucacuucuaugcaagucagcuugcacaaaggaaaauucaggaaauauuggcucagguaagaagacagcagcagcaacagcagaaaacagugcaaaguggacaaccccaaccgagaagaaaauaaagguucaggaaaauggcccuucacagagacagaugccagaccaaagacagacugagaugaagacaaauuccacuucuugucccugcuaugugggaagaauaccaccuagcgcauccccuguuugaggaccaggcaaccauuaaacugucucugagaauguauuauuuaggcuuucaaaacuuuugaaacccagcuuuaaaaauggaaaaaaaggcaaaaaaagaaaaaaaaaaaaaaaaaaa

SEQ ID NO: 6 - protein, 577 aa, Homo sapiens
  ref <308>: DATABASE ACCESSION NUMBER: NP_006537
  ref <309>: DATABASE ENTRY DATE: 2005-07-13
  ref <313>: RELEVANT RESIDUES: (1)..(577)
MNKLYIGNLNESVTPADLEKVFAEHKISYSGQFLVKSGYAFVDCPDEHWAMKAIETFSGKVELQGKRLEIEHSVPKKQRSRKIQIRNIPPQLRWEVLDSLLAQYGTVENCEQVNTESETAVVNVTYSNREQTRQAIMKLNGHQLENHALKVSYIPDEQIAQGPENGRRGGFGSRGQPRQGSPVAAGAPAKQQQVDIPLRLLVPTQYVGAIIGKEGATIRNITKQTQSKIDVHRKENAGAAEKAISVHSTPEGCSSACKMILEIMHKEAKDTKTADEVPLKILAHNNFVGRLIGKEGRNLKKVEQDTETKITISSLQDLTLYNPERTITVKGAIENCCRAEQEIMKKVREAYENDVAAMSLQSHLIPGLNLAAVGLFPASSSAVPPPPSSVTGAAPYSSFMQAPEQEMVQVFIPAQAVGAIIGKKGQHIKQLSRFASASIKIAPPETPDSKVRMVIITGPPEAQFKAQGRIYGKLKEENFFGPKEEVKLETHIRVPASAAGRVIGKGGKTVNELQNLTAAEVVVPRDQTPDENDQVIVKIIGHFYASQMAQRKIRDILAQVKQQHQKGQSNQAQARRK

SEQ ID NO: 7 - DNA, 8769 bp, Homo sapiens
  ref <308>: DATABASE ACCESSION NUMBER: NM_006546
  ref <309>: DATABASE ENTRY DATE: 2005-07-13
  ref <313>: RELEVANT RESIDUES: (1)..(8769)
atttagaggcggcgccagggcggccgcggagaaacgtgacacaccagccctctcggaggggtttcggaccgaagggaagaagctgcgccgtgtcgtccgtctccctgcgcgccgcgggcacttctcctgggctctccccgaactctcccgcgacctctgcgcgccctcaggccgccttccccgccctgggctcgggacaacttctggggtggggtgcaaagaaagtttgcggctcctgccgccggcctctccgcctcttggcctaggaggctcgccgcccgcgcccgctcgttcggccttgcccgggaccgcgtcctgccccgagaccgccaccatgaacaagctttacatcggcaacctcaacgagagcgtgacccccgcggacttggagaaagtgtttgcggagcacaagatctcctacagcggccagttcttggtcaaatccggctacgccttcgtggactgcccggacgagcactgggcgatgaaggccatcgaaactttctccgggaaagtagaattacaaggaaaacgcttagagattgaacattcggtgcccaaaaaacaaaggagccggaaaattcaaatccgaaatattccaccccagctccgatgggaagtactggacagcctgctggctcagtatggtacagtagagaactgtgagcaagtgaacaccgagagtgagacggcagtggtgaatgtcacctattccaaccgggagcagaccaggcaagccatcatgaagctgaatggccaccagttggagaaccatgccctgaaggtctcctacatccccgatgagcagatagcacagggacctgagaatgggcgccgagggggctttggctctcggggtcagccccgccagggctcacctgtggcagcgggggccccagccaagcagcagcaagtggacatcccccttcggctcctggtgcccacccagtatgtgggtgccattattggcaaggagggggccaccatccgcaacatcacaaaacagacccagtccaagatagacgtgcataggaaggagaacgcaggtgcagctgaaaaagccatcagtgtgcactccacccctgagggctgctcctccgcttgtaagatgatcttggagattatgcataaagaggctaaggacaccaaaacggctgacgaggttcccctgaagatcctggcccataataactttgtagggcgtctcattggcaaggaaggacggaacctgaagaaggtagagcaagataccgagacaaaaatcaccatctcctcgttgcaagaccttaccctttacaaccctgagaggaccatcactgtgaagggggccatcgagaattgttgcagggccgagcaggaaataatgaagaaagttcgggaggcctatgagaatgatgtggctgccatgagcctgcagtctcacctgatccctggcctgaacctggctgctgtaggtcttttcccagcttcatccagcgcagtcccgccgcctcccagcagcgttactggggctgctccctatagctcctttatgcaggctcccgagcaggagatggtgcaggtgtttatccccgcccaggcagtgggcgccatcatcggcaagaaggggcagcacatcaaacagctctcccggtttgccagcgcctccatcaagattgcaccacccgaaacacctgactccaaagttcgtatggttatcatcactggaccgccagaggcccaattcaaggctcagggaagaatctatggcaaactcaaggaggagaacttctttggtcccaaggaggaagtgaagctggagacccacatacgtgtgccagcatcagcagctggccgggtcattggcaaaggtggaaaaacggtgaacgagttgcagaatttgacggcagctgaggtggtagtaccaagagaccagacccctgatgagaacgaccaggtcatcgtgaaaatcatcggacatttctatgccagtcagatggctcaacggaagatccgagacatcctggcccaggttaagcagcagcatcagaagggacagagtaaccaggcccaggcacggaggaagtgaccagcccctccctgtcccttcgagtccaggacaacaacgggcagaaatcgagagtgtgctctccccggcaggcctgagaatgagtgggaatccgggacacctgggccgggctgtagatcaggtttgcccacttgattgagaaagatgttccagtgaggaaccctgatctctcagccccaaacacccacccaattggcccaacactgtctgcccctcggggtgtcagaaattctagcgcaaggcacttttaaacgtggattgtttaaagaagctctccaggccccaccaagagggtggatcacacctcagtgggaagaaaaataaaatttccttcaggttttaaaaacatgcagagaggtgttttaatcagccttaaaggatggttcatttcttgaccttaatgtttttccaatcttcttccccctacttgggtaattgattaaaatacctccatttacggcctctttctatatttacactaatttttttatctttattgctaccagaaaaaaatgcgaacgaatgcattgctttgcttacagtattgactcaagggaaaagaactgtcagtatctgtagattaattccaatcactccctaaccaataggtacaatacggaatgaagaagaggggaaaatggggagaaagatggttaaaatacataataatccacgtttaaaaggagcgcacttgtggctgatctatgccagatcaccatcttcaaattggcacaactgaaatttccccactctgttggggcttccccaccacattcatgtccctctcccgtgtaggtttcacattatgtccaggtgcacataggtggtattgaatgctcagcagggtaggggctgaccactgtccctgattcccatcgttctcaggcggattttatatttttttaaagtctattttaatgattggatatgagcactgggaaggggacgctaactccccttgataaagtctcggttccatggaggacttgagtggccccaaaggctgccacggtgccctcaccccagcccatgtgctcccataagggctggttcctagaggcaggggttgtggggcactcccagccacggcactgttaccttggtggtgggacttggaacccaaccctgagctcccgataaagctaaagtccatcatctggcaaattcagtaaattggagagtacttgcttctgtttgtatctgagaggaatttttaactgacggcttctgtctccatgaatcattatcagcatgatgaaaggtgtgtctaaaaaacaattcagaataccagcagcattgtacagcaaggggtaaataagcttaatttattaatttaccaggcttaattaagatcccatggagtgtttagcccttgtgggagacagaagccatcagttaaatgaggttaggcctctcctcctaatatactgattgacaatgcatattagccaggtaatgcactttagctaccctggacaatgctatcaagtgtgctgggaagggaggaaggcctctctacatatggaaaagcccatgcgtggagttcccctcctttcaacattgcaacaacagtaacaacaagacaaccgcaacatgtgggcgtagtcaggcaatgctgtgtgcgaagtaaactacctcaaggtatgaagttacctcagcaattattttcctttttgttccccccaaccccattaaaaaaatttttttttgatttttgtttttttgcagcttgctgatattttatataaaaaagaaaagcaaagcaaaagagaagctgatagtcttgaatattttatttttttaatgaaaagaaaaaacaagaaagttatgtttcataatttcttacaacatgagccagtaaccctttaggaactctctatggagaacaggcctggtgggaaaggctttgggggctgcccccttaggaggaggctagtgctaagagggaaggcccaggtttgagagagcccagaggggcagagcccagagccttgtttggccctgatctctgacttctagagccccagctgctggcggctgctggaatatcctacctgataggattaaaaggcctagtggagctgggggctctcagtggttaaacaatgcccaacaaccaaccagctggcccttggtctcctctctttcctcctttggttaaagagcatctcagccagcttttcccaccagtggtgctgttgagatattttaaaatattgcctccgttttatcgaggagagaaataataactaaaaaatataccctttaaaaaaacctatatttctctgtctaaaaatatgggagctgagattccgttcgtggaaaaaagacaaggccaccctctcgccctcagagaggtccacctggtttgtcattgcaatgcttttcattttttttttttgttattgtttcatttcagttccgtcttgctattcttcctaatctatatccatagatctaaggggcaaacagatactagttaactgcccccacctctgtctccctgtcttctttagatcggtctgattgattttaaaagtggacccaaacttagggaattcttgatttagggtggctggtggcaaggaggggcaggggatatggggacgtgactgggacaggttcctgccttatcattttctccctaggacattcccttgtagcccccagaattgtctggcccaaattgaatagaagcagaaaaacatttagggataacatcaggccagtagaattaagcctctccacctgtcccaaccataaaaagggtctcccagctttccatctctggctctatatgctttatcccaaaacaaagcagataacgttcagacgtcggccatttagtaatttaaagcgaatttccagcagcaagcatgctttgatatctggttcagactatcatcaggaagaaaaaaaaatcccacagtacctgaaatgtgattgttgcagtgttcagtttccttgggggcctgctcccttcacaccttgagcccaagtccttttccgttggctgattcagctcccagaagagacgaggaagtgtgtggcaagggactggaaaacttcacttgcttggattaggcaaggctccactcattgttgatatttgcccagcaggaaaatcatgtaagttataccaccagaaagcaaaaggagcatggtttggtggttaaggtttagtgggatgaaggacctgtcttggtgggccgggccctcttgtgccccgtaggctaggtcttagggcaactccttgccctcctgctcagcacctccatttccccatccttggtgagataacaagctatcgcgaaaagcacttgggagatttggatgatttgagaagagtgacttaaaaaaaatgcttctgtgctctaagatatatatgtgtgtgtgtgtgctacatatatatttttaagaaaggaccatctctttaggatatatttttaaattctttgaaacacataaccaaaatggtttgattcactgactgactttgaagctgcatctgccagttacaccccaaatggctttaatcccctctcgggtctggttgccttttgcagtttgggttgtggactcagctcctgtgaggggtctggttaggagagagccatttttaaggacagggagttttatagcccttttctactttcctcccctcctcccagtccttatcaatcttttttcctttttcctgaccccctccttctggaggcagttgggagctatccttgtttatgcctcactattggcagaaaagaccccatttaaaacccagagaacactggagggggatgctctagttggttctgtgtccattttcctctgtgccaaagacagacagacagaggctgagagaggctgttcctgaatcaaagcaatagccagctttcgacacatacctggctgtctgaggaggaaggcctcctggaaactgggagctaagggcgaggcccttcccttcagaggctcctgggggattagggtgtggtgtttgccaagccaaggggtagggagccgagaaattggtctgtcggctcctggttgcactttggggaaggagaggaagtttggggctccaggtagctccctgttgtgggactgctctgtcccctgcccctactgcagagatagcactgccgagttcccttcaggcctggcagacgggcagtgaggaggggcctcagttagctctcaagggtgccttcccctcctcccaacccagacataccctctgccaaactgggaaccagcagtgctagtaactacctcacagagccccagagggcctgcttgagccttcttgctccacaggagaagctggtgcctctaggcaaccccttcctcccacctctcatcaggggtgggggttctcctttctttcccctgaagtgtttatggggagatcctagtggctttgccattcaaaccactcgactgtttgcctgtttcttgaaaaccagtagaagggaaacagcacagcctgtcacagtaattgcaggaagattgaagaaaaatcctcatcaatgccaggggacataaaagccatttcccttccaaatactcgacaatttagatgcagaacatttctctgtattcagacttagagtaacaccagctgaaaactgcagtttctttcctttggatacataaggcttctctatcggggtacgggacagggaggaggcctcatgtctgaagggggatttaggggcgagagccccagccctgaccctcggtcctgtgcaccgctttggggcacagtctgatggcgcctttgctggcgccttagtatggttgactccggatggacaaaagaaaaaaaattttttttcttgaatgaaatagcaggaagctcctcgggagcatgtgttttgattaaccgcaggtgatggatgctacgagtataaatggattaactacctcaatccttacagtaagattggaactaagggcagggactcatgcataagggtatgaatcccagccaggacaagtgagttgaggcttgtgccacaaaaggtttgtccttggggaacaggcaggcctgccaggatcccccccatatcgattgggctgggagggctggccatgaggtccccactttctgctttccttgcccatgtgtcacccctttggcctccagcttgtccctctctcactttctatagctttgttggaccagatggtgaggaaaggaatggcctcttcccttctagagggggctggctggagtgagacctggggcttggcctggaacccaccacacagccccaaagtcaggaagcctggggaaaccagagctgagacctcttcaacagggtttctttgagatcctacacctccattgggccctttttcagtcttcaatgggggcccagttggctctagaaggagaagaggtgaagcaggatcctttgccctgggggagtctgagggcgcggtccttggactcattcaggccgtctttgtagttgggggagttccactgggcgatcccagcccctccccacccaccctctaatggacctcctcatagaagccccatttcacttttgttttatctacctcttagcaaaacaatagataaattaggtagtggcagctccacttgcttaggttagggggggaaaaagatttctttttccaaaggaaaaaaatattaccttgagaatactttccaaaaaataaaattaaaaaaaaaaaaaccaaaaaaaaaaatttttttttaaaagggagacattttccagtgaccactggattgttttaatttcccaagcttttttttcccccataaataagtttcactctttggcgattttcttcacttgtttaagataacgtgctagctattccaacaggtaacagctttcacagtctgcccctggcctgtctcaccccatcccccaccctattcctgccagtgagtccttcctgtgcttctctcccttctcccctcccagccagctgacttcagtcacccctgtcccccctcccctgccaataagctcccccaggaataaaggctttgttttggggatgcttaaatcttgactggcacttcccggctgtgggggctggggagccacttgtaacatttctgtgcagattttatgttagccactgctatgtaaaagcacgttcaaaatgaatttcagcagattatgtgttaccataatgaataaacgtcctctatcaccatttggagtctcccttttctccaggatcttgatcctggtccccaaaaccagagtgaatcaaaagagcttcctcccctgaggcaaagtggatttgtaagcagttctgaaacatcacttactcagaagagggaacgatgtattttgatgagtgcaaattgggaagagctggaggcctactgcttgggacagttttttttttttttttttttttaaatatgagtgctagcttattctgtaattgcggcaactttgaaaattgtattttactggaaatctgccagccatcaccacccgattttgattgtatccttcctcccatcctttaatctgttcattgctttgggggaggtggggcagctggctcacacgttggagtttgttctttgatggatgaacgaacactccagttttctttcccgtgaaggttgtttcagccacaaaccacttcattttgctgtttcaatttcaaaataaaaggaaacttatattgaaagacaa

SEQ ID NO: 8 - protein, 599 aa, Homo sapiens
  ref <308>: DATABASE ACCESSION NUMBER: NP_006539
  ref <309>: DATABASE ENTRY DATE: 2005-06-03
  ref <313>: RELEVANT RESIDUES: (1)..(599)
MMNKLYIGNLSPAVTADDLRQLFGDRKLPLAGQVLLKSGYAFVDYPDQNWAIRAIETLSGKVELHGKIMEVDYSVSKKLRSRKIQIRNIPPHLQWEVLDGLLAQYGTVENVEQVNTDTETAVVNVTYATREEAKIAMEKLSGHQFENYSFKISYIPDEEVSSPSPPQRAQRGDHSSREQGHAPGGTSQARQIDFPLRILVPTQFVGAIIGKEGLTIKNITKQTQSRVDIHRKENSGAAEKPVTIHATPEGTSEACRMILEIMQKEADETKLAEEIPLKILAHNGLVGRLIGKEGRNLKKIEHETGTKITISSLQDLSIYNPERTITVKGTVEACASAEIEIMKKLREAFENDMLAVNQQANLIPGLNLSALGIFSTGLSVLSPPAGPRGAPPAAPYHPFTTHSGYFSSLYPHHQFGPFPHHHSYPEQEIVNLFIPTQAVGAIIGKKGAHIKQLARFAGASIKIAPAEGPDVSERMVIITGPPEAQFKAQGRIFGKLKEENFFNPKEEVKLEAHIRVPSSTAGRVIGKGGKTVNELQNLTSAEVIVPRDQTPDENEEVIVRIIGHFFASQTAQRKIREIVQQVKQQEQKYPQGVASQRSK

SEQ ID NO: 9 - DNA, 3676 bp, Homo sapiens
  ref <308>: DATABASE ACCESSION NUMBER: NM_006548
  ref <309>: DATABASE ENTRY DATE: 2005-06-03
  ref <313>: RELEVANT RESIDUES: (1)..(3676)
gagagacgagggcagcggaggaggcgaggagcgccgggtaccgggccgggggagccgcgggctctcggggaagagacggatgatgaacaagctttacatcgggaacctgagccccgccgtcaccgccgacgacctccggcagctctttggggacaggaagctgcccctggcgggacaggtcctgctgaagtccggctacgccttcgtggactaccccgaccagaactgggccatccgcgccatcgagaccctctcgggtaaagtggaattgcatgggaaaatcatggaagttgattactcagtctctaaaaagctaaggagcaggaaaattcagattcgaaacatccctcctcacctgcagtgggaggtgttggatggacttttggctcaatatgggacagtggagaatgtggaacaagtcaacacagacacagaaaccgccgttgtcaacgtcacatatgcaacaagagaagaagcaaaaatagccatggagaagctaagcgggcatcagtttgagaactactccttcaagatttcctacatcccggatgaagaggtgagctccccttcgccccctcagcgagcccagcgtggggaccactcttcccgggagcaaggccacgcccctgggggcacttctcaggccagacagattgatttcccgctgcggatcctggtccccacccagtttgttggtgccatcatcggaaaggagggcttgaccataaagaacatcactaagcagacccagtcccgggtagatatccatagaaaagagaactctggagctgcagagaagcctgtcaccatccatgccaccccagaggggacttctgaagcatgccgcatgattcttgaaatcatgcagaaagaggcagatgagaccaaactagccgaagagattcctctgaaaatcttggcacacaatggcttggttggaagactgattggaaaagaaggcagaaatttgaagaaaattgaacatgaaacagggaccaagataacaatctcatctttgcaggatttgagcatatacaacccggaaagaaccatcactgtgaagggcacagttgaggcctgtgccagtgctgagatagagattatgaagaagctgcgtgaggcctttgaaaatgatatgctggctgttaaccaacaagccaatctgatcccagggttgaacctcagcgcacttggcatcttttcaacaggactgtccgtgctatctccaccagcagggccccgcggagctccccccgctgccccctaccaccccttcactacccactccggatacttctccagcctgtacccccatcaccagtttggcccgttcccgcatcatcactcttatccagagcaggagattgtgaatctcttcatcccaacccaggctgtgggcgccatcatcgggaagaagggggcacacatcaaacagctggcgagattcgccggagcctctatcaagattgcccctgcggaaggcccagacgtcagcgaaaggatggtcatcatcaccgggccaccggaagcccagttcaaggcccagggacggatctttgggaaactgaaagaggaaaacttctttaaccccaaagaagaagtgaagctggaagcgcatatcagagtgccctcttccacagctggccgggtgattggcaaaggtggcaagaccgtgaacgaactgcagaacttaaccagtgcagaagtcatcgtgcctcgtgaccaaacgccagatgaaaatgaggaagtgatcgtcagaattatcgggcacttctttgctagccagactgcacagcgcaagatcagggaaattgtacaacaggtgaagcagcaggagcagaaataccctcagggagtcgcctcacagcgcagcaagtgaggctcccacaggcaccagcaaaacaacggatgaatgtagcccttccaacacctgacagaatgagaccaaacgcagccagccagatcgggagcaaaccaaagaccatctgaggaatgagaagtctgcggaggcggccagggactctgccgaggccctgagaaccccaggggccgaggaggggcggggaaggtcagccaggtttgccagaaccaccgagccccgcctcccgccccccagggcttctgcaggcttcagccatccacttcaccatccactcggatctctcctgaactcccacgacgctatcccttttagttgaactaacataggtgaacgtgttcaaagccaagcaaaatgcacaccctttttctgtggcaaatcgtctctgtacatgtgtgtacatattagaaagggaagatgttaagatatgtggcctgtgggttacacagggtgcctgcagcggtaatatattttagaaataatatatcaaataactcaactaactccaatttttaatcaattattaatttttttttctttttaaagagaaagcaggcttttctagactttaaagaataaagtctttgggaggtctcacggtgtagagaggagctttgaggccacccgcacaaaattcacccagagggaaatctcgtcggaaggacactcacggcagttctggatcacctgtgtatgtcaacagaagggataccgtctccttgaagaggaaactctgtcactcctcatgcctgtctagctcatacacccatttctctttgcttcacaggttttaaactggttttttgcatactgctatataattctctgtctctctctgtttatctctcccctccctcccctccccttcttctccatctccattcttttgaatttcctcatccctccatctcaatcccgtatctacgcacccccccccccccaggcaaagcagtgctctgagtatcacatcacacaaaaggaacaaaagcgaaacacacaaaccagcctcaacttacacttggttactcaaaagaacaagagtcaatggtacttgtcctagcgttttggaagaggaaaacaggaacccaccaaaccaaccaatcaaccaaacaaagaaaaaattccacaatgaaagaatgtattttgtctttttgcattttggtgtataagccatcaatattcagcaaaatgattcctttctttaaaaaaaaaaaatgtggaggaaagtagaaatttaccaaggttgttggcccagggcgttaaattcacagatttttttaacgagaaaaacacacagaagaagctacctcaggtgtttttacctcagcaccttgctcttgtgtttcccttagagattttgtaaagctgatagttggagcatttttttatttttttaataaaaatgagttggaaaaaaaataagatatcaactgccagcctggagaaggtgacagtccaagtgtgcaacagctgttctgaattgtcttccgctagccaagaacctatatggccttcttttggacaaaccttgaaaatgtttatttaaaaaaaaaaaagatgacaaagaaaaacagagagagagaatattggagatgtcctgaattttaatagggtacgcgccattagggctttttgcgctaaaggatgaacatgtactggtttatgtggacaagccattataccaccagactgcaatgccagtttcctctactgcaaacagtgttctgtgacaaaaaaaaaaaaaaaaaaaagaaaaaaaaagaaaaaacagaaatatatccagctaacaagaaaaaa

SEQ ID NO: 10 - protein, 556 aa, Homo sapiens
  ref <308>: DATABASE ACCESSION NUMBER: NP_001007226
  ref <309>: DATABASE ENTRY DATE: 2005-06-03
  ref <313>: RELEVANT RESIDUES: (1)..(556)
MMNKLYIGNLSPAVTADDLRQLFGDRKLPLAGQVLLKSGYAFVDYPDQNWAIRAIETLSGKVELHGKIMEVDYSVSKKLRSRKIQIRNIPPHLQWEVLDGLLAQYGTVENVEQVNTDTETAVVNVTYATREEAKIAMEKLSGHQFENYSFKISYIPDEEVSSPSPPQRAQRGDHSSREQGHAPGGTSQARQIDFPLRILVPTQFVGAIIGKEGLTIKNITKQTQSRVDIHRKENSGAAEKPVTIHATPEGTSEACRMILEIMQKEADETKLAEEIPLKILAHNGLVGRLIGKEGRNLKKIEHETGTKITISSLQDLSIYNPERTITVKGTVEACASAEIEIMKKLREAFENDMLAVNTHSGYFSSLYPHHQFGPFPHHHSYPEQEIVNLFIPTQAVGAIIGKKGAHIKQLARFAGASIKIAPAEGPDVSERMVIITGPPEAQFKAQGRIFGKLKEENFFNPKEEVKLEAHIRVPSSTAGRVIGKGGKTVNELQNLTSAEVIVPRDQTPDENEEVIVRIIGHFFASQTAQRKIREIVQQVKQQEQKYPQGVASQRSK

SEQ ID NO: 11 - DNA, 3547 bp, Homo sapiens
  ref <308>: DATABASE ACCESSION NUMBER: NM_001007225
  ref <309>: DATABASE ENTRY DATE: 2005-06-03
  ref <313>: RELEVANT RESIDUES: (1)..(3547)
gagagacgagggcagcggaggaggcgaggagcgccgggtaccgggccgggggagccgcgggctctcggggaagagacggatgatgaacaagctttacatcgggaacctgagccccgccgtcaccgccgacgacctccggcagctctttggggacaggaagctgcccctggcgggacaggtcctgctgaagtccggctacgccttcgtggactaccccgaccagaactgggccatccgcgccatcgagaccctctcgggtaaagtggaattgcatgggaaaatcatggaagttgattactcagtctctaaaaagctaaggagcaggaaaattcagattcgaaacatccctcctcacctgcagtgggaggtgttggatggacttttggctcaatatgggacagtggagaatgtggaacaagtcaacacagacacagaaaccgccgttgtcaacgtcacatatgcaacaagagaagaagcaaaaatagccatggagaagctaagcgggcatcagtttgagaactactccttcaagatttcctacatcccggatgaagaggtgagctccccttcgccccctcagcgagcccagcgtggggaccactcttcccgggagcaaggccacgcccctgggggcacttctcaggccagacagattgatttcccgctgcggatcctggtccccacccagtttgttggtgccatcatcggaaaggagggcttgaccataaagaacatcactaagcagacccagtcccgggtagatatccatagaaaagagaactctggagctgcagagaagcctgtcaccatccatgccaccccagaggggacttctgaagcatgccgcatgattcttgaaatcatgcagaaagaggcagatgagaccaaactagccgaagagattcctctgaaaatcttggcacacaatggcttggttggaagactgattggaaaagaaggcagaaatttgaagaaaattgaacatgaaacagggaccaagataacaatctcatctttgcaggatttgagcatatacaacccggaaagaaccatcactgtgaagggcacagttgaggcctgtgccagtgctgagatagagattatgaagaagctgcgtgaggcctttgaaaatgatatgctggctgttaacacccactccggatacttctccagcctgtacccccatcaccagtttggcccgttcccgcatcatcactcttatccagagcaggagattgtgaatctcttcatcccaacccaggctgtgggcgccatcatcgggaagaagggggcacacatcaaacagctggcgagattcgccggagcctctatcaagattgcccctgcggaaggcccagacgtcagcgaaaggatggtcatcatcaccgggccaccggaagcccagttcaaggcccagggacggatctttgggaaactgaaagaggaaaacttctttaaccccaaagaagaagtgaagctggaagcgcatatcagagtgccctcttccacagctggccgggtgattggcaaaggtggcaagaccgtgaacgaactgcagaacttaaccagtgcagaagtcatcgtgcctcgtgaccaaacgccagatgaaaatgaggaagtgatcgtcagaattatcgggcacttctttgctagccagactgcacagcgcaagatcagggaaattgtacaacaggtgaagcagcaggagcagaaataccctcagggagtcgcctcacagcgcagcaagtgaggctcccacaggcaccagcaaaacaacggatgaatgtagcccttccaacacctgacagaatgagaccaaacgcagccagccagatcgggagcaaaccaaagaccatctgaggaatgagaagtctgcggaggcggccagggactctgccgaggccctgagaaccccaggggccgaggaggggcggggaaggtcagccaggtttgccagaaccaccgagccccgcctcccgccccccagggcttctgcaggcttcagccatccacttcaccatccactcggatctctcctgaactcccacgacgctatcccttttagttgaactaacataggtgaacgtgttcaaagccaagcaaaatgcacaccctttttctgtggcaaatcgtctctgtacatgtgtgtacatattagaaagggaagatgttaagatatgtggcctgtgggttacacagggtgcctgcagcggtaatatattttagaaataatatatcaaataactcaactaactccaatttttaatcaattattaatttttttttctttttaaagagaaagcaggcttttctagactttaaagaataaagtctttgggaggtctcacggtgtagagaggagctttgaggccacccgcacaaaattcacccagagggaaatctcgtcggaaggacactcacggcagttctggatcacctgtgtatgtcaacagaagggataccgtctccttgaagaggaaactctgtcactcctcatgcctgtctagctcatacacccatttctctttgcttcacaggttttaaactggttttttgcatactgctatataattctctgtctctctctgtttatctctcccctccctcccctccccttcttctccatctccattcttttgaatttcctcatccctccatctcaatcccgtatctacgcacccccccccccccaggcaaagcagtgctctgagtatcacatcacacaaaaggaacaaaagcgaaacacacaaaccagcctcaacttacacttggttactcaaaagaacaagagtcaatggtacttgtcctagcgttttggaagaggaaaacaggaacccaccaaaccaaccaatcaaccaaacaaagaaaaaattccacaatgaaagaatgtattttgtctttttgcattttggtgtataagccatcaatattcagcaaaatgattcctttctttaaaaaaaaaaaatgtggaggaaagtagaaatttaccaaggttgttggcccagggcgttaaattcacagatttttttaacgagaaaaacacacagaagaagctacctcaggtgtttttacctcagcaccttgctcttgtgtttcccttagagattttgtaaagctgatagttggagcatttttttatttttttaataaaaatgagttggaaaaaaaataagatatcaactgccagcctggagaaggtgacagtccaagtgtgcaacagctgttctgaattgtcttccgctagccaagaacctatatggccttcttttggacaaaccttgaaaatgtttatttaaaaaaaaaaaagatgacaaagaaaaacagagagagagaatattggagatgtcctgaattttaatagggtacgcgccattagggctttttgcgctaaaggatgaacatgtactggtttatgtggacaagccattataccaccagactgcaatgccagtttcctctactgcaaacagtgttctgtgacaaaaaaaaaaaaaaaaaaaagaaaaaaaaagaaaaaacagaaatatatccagctaacaagaaaaaa

SEQ ID NO: 12 - protein, 579 aa, Homo sapiens
  ref <308>: DATABASE ACCESSION NUMBER: NP_006538
  ref <309>: DATABASE ENTRY DATE: 2005-04-23
  ref <313>: RELEVANT RESIDUES: (1)..(579)
MNKLYIGNLSENAAPSDLESIFKDAKIPVSGPFLVKTGYAFVDCPDESWALKAIEALSGKIELHGKPIEVEHSVPKRQRIRKLQIRNIPPHLQWEVLDSLLVQYGVVESCEQVNTDSETAVVNVTYSSKDQARQALDKLNGFQLENFTLKVAYIPDEMAAQQNPLQQPRGRRGLGQRGSSRQGSPGSVSKQKPCDLPLRLLVPTQFVGAIIGKEGATIRNITKQTQSKIDVHRKENAGAAEKSITILSTPEGTSAACKSILEIMHKEAQDIKFTEEIPLKILAHNNFVGRLIGKEGRNLKKIEQDTDTKITISPLQELTLYNPERTITVKGNVETCAKAEEEIMKKIRESYENDIASMNLQAHLIPGLNLNALGLFPPTSGMPPPTSGPPSAMTPPYPQFEQSETETVHLFIPALSVGAIIGKQGQHIKQLSRFAGASIKIAPAEAPDAKVRMVIITGPPEAQFKAQGRIYGKIKEENFVSPKEEVKLEAHIRVPSFAAGRVIGKGGKTVNELQNLSSAEVVVPRDQTPDENDQVVVKITGHFYACQVAQRKIQEILTQVKQHQQQKALQSGPPQSRRK

SEQ ID NO: 13 - DNA, 4168 bp, Homo sapiens
  ref <308>: DATABASE ACCESSION NUMBER: NM_006547
  ref <309>: DATABASE ENTRY DATE: 2005-04-23
  ref <313>: RELEVANT RESIDUES: (1)..(4168)
aagacttaggaagactggtggatgcgtttgggttgtagctaggctttttcttttctttctcttttaaaacacatctagacaaggaaaaaacaagcctcggatctgatttttcactcctcgttcttgtgcttggttcttactgtgtttgtgtattttaaaggcgagaagacgaggggaacaaaaccagctggatccatccatcaccgtgggtggttttaatttttcgttttttctcgttatttttttttaaacaaccactcttcacaatgaacaaactgtatatcggaaacctcagcgagaacgccgccccctcggacctagaaagtatcttcaaggacgccaagatcccggtgtcgggacccttcctggtgaagactggctacgcgttcgtggactgcccggacgagagctgggccctcaaggccatcgaggcgctttcaggtaaaatagaactgcacgggaaacccatagaagttgagcactcggtcccaaaaaggcaaaggattcggaaacttcagatacgaaatatcccgcctcatttacagtgggaggtgctggatagtttactagtccagtatggagtggtggagagctgtgagcaagtgaacactgactcggaaactgcagttgtaaatgtaacctattccagtaaggaccaagctagacaagcactagacaaactgaatggatttcagttagagaatttcaccttgaaagtagcctatatccctgatgaaatggccgcccagcaaaaccccttgcagcagccccgaggtcgccgggggcttgggcagaggggctcctcaaggcaggggtctccaggatccgtatccaagcagaaaccatgtgatttgcctctgcgcctgctggttcccacccaatttgttggagccatcataggaaaagaaggtgccaccattcggaacatcaccaaacagacccagtctaaaatcgatgtccaccgtaaagaaaatgcgggggctgctgagaagtcgattactatcctctctactcctgaaggcacctctgcggcttgtaagtctattctggagattatgcataaggaagctcaagatataaaattcacagaagagatccccttgaagattttagctcataataactttgttggacgtcttattggtaaagaaggaagaaatcttaaaaaaattgagcaagacacagacactaaaatcacgatatctccattgcaggaattgacgctgtataatccagaacgcactattacagttaaaggcaatgttgagacatgtgccaaagctgaggaggagatcatgaagaaaatcagggagtcttatgaaaatgatattgcttctatgaatcttcaagcacatttaattcctggattaaatctgaacgccttgggtctgttcccacccacttcagggatgccacctcccacctcagggcccccttcagccatgactcctccctacccgcagtttgagcaatcagaaacggagactgttcatctgtttatcccagctctatcagtcggtgccatcatcggcaagcagggccagcacatcaagcagctttctcgctttgctggagcttcaattaagattgctccagcggaagcaccagatgctaaagtgaggatggtgattatcactggaccaccagaggctcagttcaaggctcagggaagaatttatggaaaaattaaagaagaaaactttgttagtcctaaagaagaggtgaaacttgaagctcatatcagagtgccatcctttgctgctggcagagttattggaaaaggaggcaaaacggtgaatgaacttcagaatttgtcaagtgcagaagttgttgtccctcgtgaccagacacctgatgagaatgaccaagtggttgtcaaaataactggtcacttctatgcttgccaggttgcccagagaaaaattcaggaaattctgactcaggtaaagcagcaccaacaacagaaggctctgcaaagtggaccacctcagtcaagacggaagtaaaggctcaggaaacagcccaccacagaggcagatgccaaaccaaagacagattgcttaaccaacagatgggcgctgaccccctatccagaatcacatgcacaagtttttacctagccagttgtttctgaggaccaggcaacttttgaactcctgtctctgtgagaatgtatactttatgctctctgaaatgtatgacacccagctttaaaacaaacaaacaaacaaacaaaaaaagggtgggggagggagggaaagagaagagctctgcacttccctttgttgtagtctcacagtataacagatattctaattcttcttaatattcccccataatgccagaaattggcttaatgatgctttcactaaattcatcaaatagattgctcctaaatccaattgttaaaattggatcagaataattatcacaggaacttaaatgttaagccattagcatagaaaaactgttctcagttttatttttacctaacactaacatgagtaacctaagggaagtgctgaatggtgttggcaggggtattaaacgtgcatttttactcaactacctcaggtattcagtaatacaatgaaaagcaaaattgttccttttttttgaaaattttatatactttataatgatagaagtccaaccgttttttaaaaaataaatttaaaatttaacagcaatcagctaacaggcaaattaagatttttacttctggctggtgacagtaaagctggaaaattaatttcagggttttttgaggcttttgacacagttattagttaaatcaaatgttcaaaaatacggagcagtgcctagtatctggagagcagcactaccatttattctttcatttatagttgggaaagtttttgacggtactaacaaagtggtcgcaggagattttggaacggctggtttaaatggcttcaggagacttcagttttttgtttagctacatgattgaatgcataataaatgctttgtgcttctgactatcaatacctaaagaaagtgcatcagtgaagagatgcaagactttcaactgactggcaaaaagcaagctttagcttgtcttataggatgcttagtttgccactacacttcagaccaatgggacagtcatagatggtgtgacagtgtttaaacgcaacaaaaggctacatttccatggggccagcactgtcatgagcctcactaagctattttgaagatttttaagcactgataaattaaaaaaaaaaaattagactccaccttaagtagtaaagtataacaggatttctgtatactgtgcaatcagttctttgaaaaaaaagtcaaaagatagagaatacaagaaaagtttttgggatataatttgaatgactgtgaaaacatatgacctttgataacgaactcatttgctcactccttgacagcaaagcccagtacgtacaattgtgttgggtgtgggtggtctccaaggccacgctgctctctgaattgattttttgagttttgtttgtaagatgatcacagtcatgttacactgatctaaaggacatatatataaccctttaaaaaaaaaatcactgcctcattcttatttcaagatgaatttctatacagactagatgtttttctgaagatcaattagacattttgaaaatgatttaaagtgttttccttaatgttctctgaaaacaagtttcttttgtagttttaaccaaaaaagtgccctttttgtcactggattctcctagcattcatgatttttttttcatacaatgaattaaaattgctaaaatcatggactggctttctggttggatttcaggtaagatgtgtttaaggccagagcttttctcagtatttgatttttttccccaatatttgattttttaaaaatatacacataggtgctgcatttatatctgctggtttaaattctgtcatatttcacttctagccttttagtatggcaaatcatattttacttttacttaagcatttgtaatttggagtatctggtactagctaagaaataattctataattgagttttgtactcaccatatatggatcattcctcatgtataatgtgccccaaatgcagcttcattttccagataccttgacgcagaataaattttttcatcatttaggtgcaaaaaaaaaa

SEQ ID NO: 14 - DNA, 1731 bp, Homo sapiens
atgaacaagctttacatcggcaacctcaacgagagcgtgacccccgcggacttggagaaagtgtttgcggagcacaagatctcctacagcggccagttcttggtcaaatccggctacgccttcgtggactgcccggacgagcactgggcgatgaaggccatcgaaactttctccgggaaagtagaattacaaggaaaacgcttagagattgaacattcggtgcccaaaaaacaaaggagccggaaaattcaaatccgaaatattccaccccagctccgatgggaagtactggacagcctgctggctcagtatggtacagtagagaactgtgagcaagtgaacaccgagagtgagacggcagtggtgaatgtcacctattccaaccgggagcagaccaggcaagccatcatgaagctgaatggccaccagttggagaaccatgccctgaaggtctcctacatccccgatgagcagatagcacagggacctgagaatgggcgccgagggggctttggctctcggggtcagccccgccagggctcacctgtggcagcgggggccccagccaagcagcagcaagtggacatcccccttcggctcctggtgcccacccagtatgtgggtgccattattggcaaggagggggccaccatccgcaacatcacaaaacagacccagtccaagatagacgtgcataggaaggagaacgcaggtgcagctgaaaaagccatcagtgtgcactccacccctgagggctgctcctccgcttgtaagatgatcttggagattatgcataaagaggctaaggacaccaaaacggctgacgaggttcccctgaagatcctggcccataataactttgtagggcgtctcattggcaaggaaggacggaacctgaagaaggtagagcaagataccgagacaaaaatcaccatctcctcgttgcaagaccttaccctttacaaccctgagaggaccatcactgtgaagggggccatcgagaattgttgcagggccgagcaggaaataatgaagaaagttcgggaggcctatgagaatgatgtggctgccatgagcctgcagtctcacctgatccctggcctgaacctggctgctgtaggtcttttcccagcttcatccagcgcagtcccgccgcctcccagcagcgttactggggctgctccctatagctcctttatgcaggctcccgagcaggagatggtgcaggtgtttatccccgcccaggcagtgggcgccatcatcggcaagaaggggcagcacatcaaacagctctcccggtttgccagcgcctccatcaagattgcaccacccgaaacacctgactccaaagttcgtatggttatcatcactggaccgccagaggcccaattcaaggctcagggaagaatctatggcaaactcaaggaggagaacttctttggtcccaaggaggaagtgaagctggagacccacatacgtgtgccagcatcagcagctggccgggtcattggcaaaggtggaaaaacggtgaacgagttgcagaatttgacggcagctgaggtggtagtaccaagagaccagacccctgatgagaacgaccaggtcatcgtgaaaatcatcggacatttctatgccagtcagatggctcaacggaagatccgagacatcctggcccaggttaagcagcagcatcagaagggacagagtaaccaggcccaggcacggaggaag

SEQ ID NO: 15 - protein, 527 aa, Homo sapiens
MNKLYIGNLNESVTPADLEKVFAEHKISYSGQFLVKSGYAFVDCPDEHWAMKAIETFSGKVELQGKRLEIEHSVPKKQRSRKIQIRNIPPQLRWEVLDSLLAQYGTVENCEQVNTESETAVVNVTYSNREQTRQAIMKLNGHQLENHALKVSYIPDEQIAQGPENGRRGGFGSRGQPRQGSPVAAGAPAKQQQVDIPLRLLVPTQYVGAIIGKEGATIRNITKQTQSKIDVHRKENAGAAEKAISVHSTPEGCSSACKMILEIMHKEAKDTKTADEVPLKILAHNNFVGRLIGKEGRNLKKVEQDTETKITISSLQDLTLYNPERTITVKGAIENCCRAEQEIMKKVREAYENDVAAMSLQSHLIPGLNLAAVGLFPASSSAVPPPPSSVTGAAPYSSFMQAPEQEMVQVFIPAQAVGAIIGKKGQHIKQLSRFASASIKIAPPETPDSKVRMVIITGPPEAQFKAQGRIYGKLKEENFFGPKEEVKLETGHFYASQMAQRKIRDILAQVKQQHQKGQSNQAQARRK

SEQ ID NO: 16 - DNA, 6392 bp, Xenopus laevis
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagcttcgaattcccatgaacaagctgtatattggaaacctcagcgaaaacgtgagccccacggatttggaaagtctcttcaaggagtctaagatccctttcaccgggcagtttctggtcaaaagtggatacgcgtttgtggactgtccggatgagacgtgggctatgaaggccatcgacaccctctcagggaaagtggagttgcatggcaaagtgattgaagtcgaacactcggtacccaaaaggcaaaggagtcgaaagcttcagatacgaaacataccgcctcatctacagtgggaggtactggacagcctgttggcacagtatgggacagtggaaaactgtgagcaagtgaacactgaatcagaaactgcagtagtgaatgtaacatatgccaataaggagcacgctagacaaggactcgaaaaattaaacggctatcagctggaaaactatagcctgaaagtgacgtatatacctgatgaaatggcaacaccgcaagcaccatcccaacagcttcagcagcagccgcagcagcaacaccctcaagggaggcgtgggtttgggcaaaggggacctgccaggcagggatcccctggtgctgctgcaagacctaagcctcagacagaggttccgctgagaatgctggttcccacacagtttgtaggggcgatcattgggaaagagggtgctaccatcaggaacatcactaaacagacccagtcaaaaatagatattcaccgtaaagaaaatgcaggtgctgcagagaaaccgattacaattcactcaacccctgaaggctgctcagctgcatgtaagatcattatggagatcatgcagaaggaagctcaggataccaagttcactgaagaaatccccttaaaaatattagcacataacaactttgttggccgtcttattggtaaagaaggaaggaaccttaagaaaattgagcaagatacagatacaaaaatcacaatatctccactacaggacttgacactgtacaatccagaacggaccattacagtaaaaggcagcattgagccatgtgccaaagccgaggaagaaatcatgaaaaaaattagggagtcctatgaaaatgatattgctgctatgaatctccaggcacatttgattcctggtttgaacctgaatgcattgggccttttcccatcatcctcatcaggaatgccaccaccttctgttggagttccctcacctacctcatctacttcttatccaccatttgggcagcagccagagtcagagaccgttcatctcttcatcccagctttagctgtgggagcgattattggaaagcaaggacaacacatcaaacaactatcacgttttgcaggagcatctattaagattgcacctgcggagggacctgatgccaaactaagaatggtgatcatcactggacctcctgaagcccaatttaaggctcaaggaaggatctatggtaaactgaaagaagaaaacttcttcggacctaaagaagaagtgaaacttgagaccggtcacttctatgcaagtcagcttgcacaaaggaaaattcaggaaatattggctcaggtaagaagacagcagcagcaacagcagaaaacagtgcaaagtggacaaccccaaccgagaagaaaataaaggggcgccccttatcactagtgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat